(19) United States
(12) Patent Application Publication
(10) Pub. No.: US 2025/0145687 A1
(43) Pub. Date: May 8, 2025

(54) MODIFIED LIGAND-GATED ION CHANNELS AND METHODS OF USE

(71) Applicant: Howard Hughes Medical Institute, Chevy Chase, MD (US)

(72) Inventor: Scott Sternson, Chevy Chase, MD (US)

(21) Appl. No.: 18/834,135

(22) PCT Filed: Jan. 31, 2023
(86) PCT No.: PCT/US2023/061662

§ 371 Date: Jul. 29, 2024

Related U.S. Application Data
(60) Provisional application No. 63/305,038, filed on Jan. 31, 2022.

(51) Int. Cl. C07K 14/705; A61K 38/00

(52) U.S. Cl. CPC C07K 14/70571; A61K 38/00; C07K 2319/02

(57) ABSTRACT

This document relates to materials and methods for modulating ligand gated ion channel (LGIC) activity. For example, modified LGICs including at least one LGIC subunit having a modified ligand binding domain (LBD) and/or a modified ion pore domain (IPD) are provided. Also provided are exogenous LGIC ligands that can bind to and activate the modified LGIC, as well as methods of modulating ion transport across the membrane of a cell of a mammal, methods of modulating the excitability of a cell in a mammal, and methods of treating a mammal having a channelopathy.

### CROSS-REFERENCE TO RELATED APPLICATION

This application claims the benefit of U.S. Patent Application Ser. No. 63/305,038, filed Jan. 31, 2022.

### BACKGROUND

### 1. Technical Field

This document relates to materials and methods for modulating ligand gated ion channel (LGIC) activity. For example, this document provides modified LGICs including at least one LGIC subunit having a modified ligand binding domain (LBD) and/or a modified ion pore domain (IPD). Also provided are exogenous LGIC ligands that can bind to and activate the modified LGIC. In some aspects, a modified LGIC and an exogenous ligand can be used to treat a mammal having a channelopathy (e.g., a neural channelopathy or a muscle channelopathy). In some aspects, a modified LGIC and an exogenous LGIC ligand can be used to modulate (e.g., activate or inhibit) ion transport across the membrane of a cell of a mammal. In some aspects, a modified LGIC and an exogenous LGIC ligand can be used to modulate (e.g., increase or decrease) the excitability of a cell in a mammal.

### 2. Background Information

Ion channels mediate ionic flux in cells, which profoundly affects their biological function. A prominent instance of this is in neurons, where ion channels control electrical signaling within and/or between neurons to influence physiology, sensation, behavior, mood, and cognition.

Different LGICs have distinct ligand binding properties as well as specific ion conductance properties (Hille 2001 Ion Channels of Excitable Membranes. pp. 814. Sunderland, MA: Sinauer Associates; Kandel et al 2000 Principles of Neural Science. USA: McGraw-Hill Co. 1414 pp). For example, nicotinic acetylcholine receptors (nAChRs) bind the endogenous ligand acetylcholine (ACh), which activates conductances for cations and typically depolarizes cells, thereby increasing cellular excitability. In contrast, the glycine receptor (GlyR) binds the endogenous ligand glycine, which activates chloride anion conductance and typically reduces the excitability of cells by hyperpolarization and/or by an electrical shunt of the cellular membrane resistance.

### SUMMARY

Levels of endogenous LGIC ligands (e.g., agonists) such as ACh are not readily controlled. For exogenous LGICs that modulate neuron activity in response to an exogenous LGIC agonist, this can lead to undesired baseline activity of these exogenous LGICs.

This document provides materials and methods for modulating LGIC activity (e.g., increasing the sensitivity of LGICs to exogenous ligands and/or reducing sensitivity to endogenous ligands such as ACh. For example, this document provides modified LGICs including at least one modified LGIC subunit having a LBD and an IPD, and having at least one modified amino acid (e.g., an amino acid substitution). Also provided are exogenous LGIC ligands that can bind to and modulate (e.g., activate) the modified LGIC. In some aspects, a modified LGIC and an exogenous ligand can be used to treat a mammal having a channelopathy (e.g., a neural channelopathy or a muscle channelopathy). In some aspects, a modified LGIC and an exogenous LGIC ligand can be used to modulate (e.g., activate or inhibit) ion transport across the membrane of a cell of a mammal. In some aspects, a modified LGIC and an exogenous LGIC ligand can be used to modulate (e.g., increase or decrease) the excitability of a cell in a mammal.

Having the ability to control LGIC activity provides a unique and unrealized opportunity to control ion transport in cells. For example, modified LGICs having increased sensitivity for one or more exogenous LGIC ligands can be used to provide temporal and spatial control of ion transport and/or cellular excitability based on delivery of the exogenous LGIC ligand. For example, modified LGICs with reduced sensitivity for endogenous LGIC ligands prevent unwanted activation of modified LGICs and allow for selective control over the modified LGIC by exogenous ligands. Further, exogenous LGIC ligands having increased potency for a modified LGIC improve selectivity of targeting of the modified LGIC over endogenous ion channels. Thus, the modified LCIGs and exogenous LGIC ligands provided herein are useful to achieve a therapeutic effect while reducing side effects from the small molecules on unintended targets.

As described herein, one or more mutations in a modified LGIC can enhance potency for exogenous LGIC ligands. Mutating residues 154, 155, 156, 163, and/or 172 of the ligand binding domain of α7 nAChR in a PSAM4-5HT3 (α7-5HT3^{L131G,Q139L,Y217F}) LGIC can reduce potency for endogenous agonists acetylcholine and choline, while maintaining high potency for exogenous agonists such as varenicline, compound 792, compound 793, and compound 817. Also as described herein, additional mutation of residue 217 in a PSAM4-5HT3 LGIC can reduce potency for endogenous agonists acetylcholine and choline, while maintaining high potency for exogenous agonists, such as compound 792, compound 793, and compound 817. These modified LGICs allow for highly selective control over cellular function in cells of a mammal while minimizing cross-reactivity with endogenous signaling systems in the mammal.

In general, one aspect of this document features modified LGICs comprising at least one modified LGIC subunit, the modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD. The amino acid substitution at residue 154 can be V154I or V154L. The amino acid substitution at residue 155 can be R155Y. The amino acid substitution at residue 156 can be W156Q or W156N. The amino acid substitution at residue 163 can be H163T, H163A, H163D, H162F, H163G, H163K, H163N, H163R, H163S, or H163V. The amino acid substitution at residue 172 can be S172A, S172V, or S172I. The modified α7-nAChR LBD also can include an amino acid substitution at residue 131 of the α7-nAChR LBD. The amino acid substitution at residue 131 can be L131A, L131G, L131M, or L131N. The modified α7-nAChR LBD also can include an amino acid substitution at residue 139 of the α7-nAChR LBD. The amino acid substitution at residue 139 can be Q139G or Q139L. The modified α7-nAChR LBD also can include an amino acid substitution at residue 217 of the α7-nAChR LBD. The amino acid substitution at residue 217 can be Y217F. The modified α7-nAChR LBD also can include a L131G amino acid substitution, a Q139L amino acid substitution, and a Y217F amino acid substitution. The IPD can be a murine 5HT3 IPD, and the murine 5HT3 IPD can include an amino acid substitution of at least one of amino acid residues 425, 429, and 433 (e.g., a R425Q substitution, a R429D substitution, and/or a R433A substitution). The IPD is a human 5HT3 IPD, and the human 5HT3 IPD can include an amino acid substitution of at least one of amino acid residues 420, 424, and 428 (e.g., a R420Q substitution, a R424D substitution, and/or a R428A substitution).

In another aspect, this document features methods for treating a channelopathy in a mammal. The methods can include, or consist essentially of, administering nucleic acid encoding a modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD to a cell in a mammal having a channelopathy under conditions in which the modified LGIC subunit can assemble into a modified LGIC comprising the modified LGIC subunit in a cell within the mammal; and administering an exogenous ligand to the mammal, where the LGIC ligand acts as an agonist of the modified LGIC. The mammal can be a human. The channelopathy can be Bartter syndrome, Brugada syndrome, catecholaminergic polymorphic ventricular tachycardia (CPVT), congenital hyperinsulinism, cystic fibrosis, Dravet syndrome, episodic ataxia, erythromelalgia, generalized epilepsy, familial hemiplegic migraine, fibromyalgia, hyperkalemic periodic paralysis, hypokalemic periodic paralysis, Lambert-Eaton myasthenic syndrome, long QT syndrome, short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, or multiple sclerosis.

In another aspect, this document features methods for modulating ion transport across a cell membrane of a mammalian cell. The methods can include, or consist essentially of, administering nucleic acid encoding a modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO: 2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO: 3, and (b) a 5HT3 IPD to a cell under conditions in which the modified LGIC subunit can assemble into a modified LGIC comprising the modified LGIC subunit within the cell; and administering an exogenous ligand to the cell, where the LGIC ligand acts as an agonist of the modified LGIC. The modulating can include activating ion transport. The modulating can include inhibiting ion transport. The cell can be a neuron, a glial cell, a myocyte, a stem cell, an endocrine cell, or an immune cell. The nucleic acid encoding the modified LGIC subunit can be administered to an in vivo cell. The nucleic acid encoding the modified LGIC subunit can be administered to an ex vivo cell. The mammalian cell can be a human cell.

In another aspect, this document features methods for modulating the activity of a cell in a mammal. The methods can include, or consist essentially of, administering nucleic acid encoding a modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD to a cell in a mammal under conditions in which the modified LGIC subunit can assemble into a modified LGIC comprising the modified LGIC subunit within the cell; and administering an exogenous ligand to the mammal, where the LGIC ligand acts as an agonist of the modified LGIC. The modulating can include increasing the activity of the cell. The modulating can include decreasing the activity of the cell. The activity of the cell can be ion transport, passive transport, excitation, inhibition, or exocytosis. The cell can be a neuron, a glial cell, a myocyte, a stem cell, an endocrine cell, or an immune cell. Administering the modified LGIC to the cell can be an in vivo administration or an ex vivo administration. Administering the modified LGIC to the cell can include administering a nucleic acid (e.g., via a viral vector such as an adeno-associated virus (AAV), a herpes simplex virus, or a lentivirus) encoding the modified LGIC.

In some aspects, the AAV capsid and/or AAV vector is of an AAV1, AAV2, AAV3, AAV4, AAV5, AAV6, AAV7, AAV8, AAVrh8, AAV9, AAVrh9, AAV10, AAVrhlO, AAV11, or AAV12 serotype. In some aspects, the AAV capsid and/or AAV vector is an AAV5 serotype.

In another aspect, this document features a mammalian cell comprising a modified LGIC comprising at least one modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO: 3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD. The mammalian cell can be a human cell. In some aspects, the modified LGIC can be administered to the mammalian cell as an in vivo administration or an ex vivo administration. Administering the modified LGIC to the cell can include administering a nucleic acid (e.g., via a viral vector such as an adeno-associated virus (AAV), a herpes simplex virus, or a lentivirus) encoding the modified LGIC.

In some aspects, the AAV capsid and/or AAV vector is of an AAV1, AAV2, AAV3, AAV4, AAV5, AAV6, AAV7, AAV8, AAVrh8, AAV9, AAVrh9, AAV10, AAVrhlO, AAV11, or AAV12 serotype. In some aspects, the AAV capsid and/or AAV vector is an AAV5 serotype.

In another aspect, this document features a nucleic acid expressing a modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD.

In another aspect, this document features a use of a modified LGIC comprising at least one modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD to treat a mammal having a channelopathy. The mammal can be a human. The channelopathy can be Bartter syndrome, Brugada syndrome, CPVT, congenital hyperinsulinism, cystic fibrosis, Dravet syndrome, episodic ataxia, erythromelalgia, generalized epilepsy, familial hemiplegic migraine, fibromyalgia, hyperkalemic periodic paralysis, hypokalemic periodic paralysis, Lambert-Eaton myasthenic syndrome, long QT syndrome, short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, or multiple sclerosis.

In another aspect, this document features a modified LGIC comprising at least one modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPDuse as a medicament to treat a mammal having a channelopathy. The mammal can be a human. The channelopathy can be Bartter syndrome, Brugada syndrome, CPVT, congenital hyperinsulinism, cystic fibrosis, Dravet syndrome, episodic ataxia, erythromelalgia, generalized epilepsy, familial hemiplegic migraine, fibromyalgia, hyperkalemic periodic paralysis, hypokalemic periodic paralysis, Lambert-Eaton myasthenic syndrome, long QT syndrome, short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, or multiple sclerosis.

In another aspect, this document features a modified LGIC comprising at least one modified LGIC subunit having (a) a modified a7-nAChR LBD, where the modified a7-nAChR LBD comprises: (i) an amino acid sequence having at least 93 percent sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a 5HT3 IPD for use in the treatment of a mammal having a channelopathy. The mammal can be a human. The channelopathy can be Bartter syndrome, Brugada syndrome, CPVT, congenital hyperinsulinism, cystic fibrosis, Dravet syndrome, episodic ataxia, erythromelalgia, generalized epilepsy, familial hemiplegic migraine, fibromyalgia, hyperkalemic periodic paralysis, hypokalemic periodic paralysis, Lambert-Eaton myasthenic syndrome, long QT syndrome, short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, or multiple sclerosis.

In some aspects, this document features an isolated cell in culture comprising a nucleic acid encoding a modified ligand gated ion channel (LGIC) comprising at least one modified LGIC subunit, the modified LGIC subunit comprising: (a) a modified alpha7 nicotinic acetylcholine receptor (a7-nAChR) ligand binding domain (LBD), wherein the modified a7-nAChR LBD comprises: (i) an amino acid sequence having at least 93% sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a serotonin 3 receptor (5HT3) ion pore domain (IPD).

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B show exemplary amino acid sequences of chimeric LGIC subunits. FIG. 1A) shows an exemplary amino acid sequence of α7-5HT3 chimeric receptor subunit (SEQ ID NO:7) including a human α7 nAChR LBD (SEQ ID NO:1) and a murine 5HT3 IPD (SEQ ID NO:5) components. FIG. 1B) shows an exemplary amino acid sequence of α7-5HT3 chimeric receptor subunit (SEQ ID NO:8) including human α7 nAChR LBD (SEQ ID NO: 1) and a human 5HT3 IPD (SEQ ID NO:6) components.

FIG. 2 shows chemical structures of exemplary LGIC agonists.

FIG. 3 is a dose response curve showing right-shifted ACh potency with additional mutations to PSAM4-5HT3.

FIGS. 4A-4D shows electrophysiological recordings in HEK 293 cells showing dose responses with right-shifted ACh potency associated with additional mutations to PSAM⁴-5HT3. FIG. 4A) corresponds to PSAM4-5HT3 with additional mutations V154I, R155Y, and W156N; FIG. 4B) corresponds to PSAM⁴-5HT3 with additional mutations V154I, R155Y, W156N, and S172A; FIG. 4C) corresponds to PSAM⁴-5HT3 (α7-5HT3^{L131G,Q139L,Y217F}); and FIG. 4D) corresponds to PSAM⁴-5HT3 with additional mutation S172A.

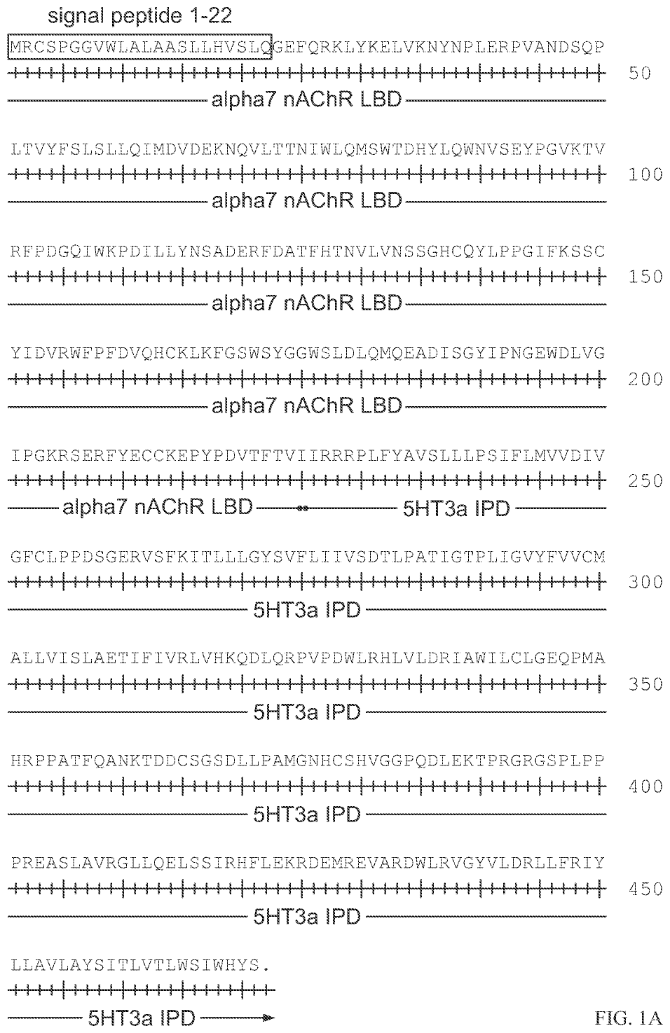

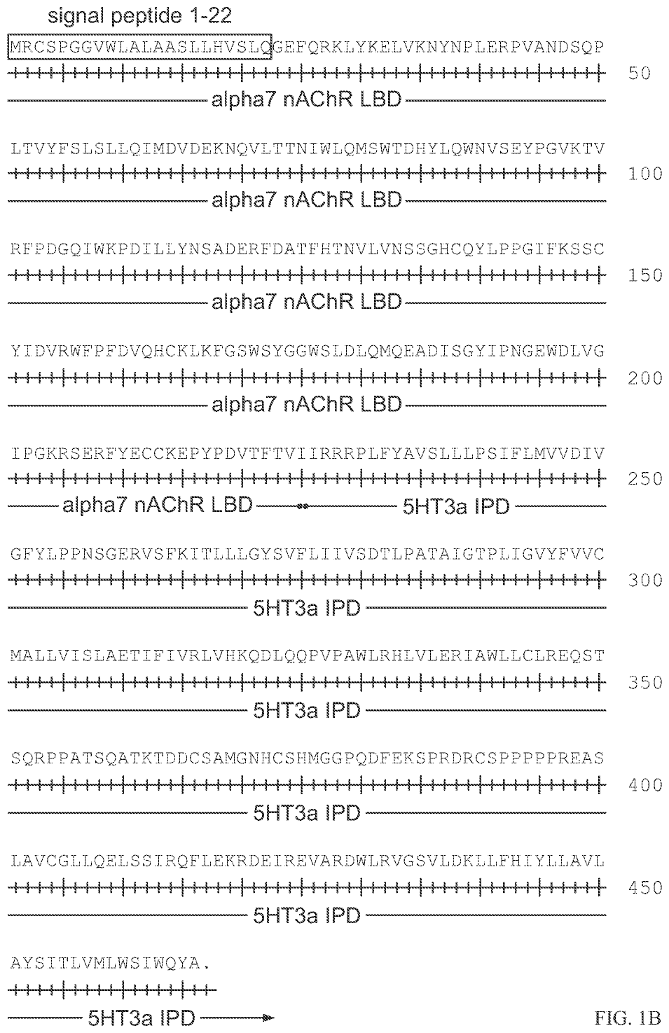

### DETAILED DESCRIPTION

This document provides modified LGICs and methods of using them. For example, this document provides modified LGICs including at least one modified LGIC subunit having a LBD and an IPD, and having at least one modified amino acid (e.g., an amino acid substitution). In some aspects, a modified LGIC provided herein can include one or more chimeric LGIC subunits. For example, a chimeric LGIC subunit can include a LBD from a first LGIC and an IPD from a second LGIC. In some aspects, the modified amino acid can confer pharmacological selectivity to the modified LGIC. For example, the modified amino acid can confer the modified LGIC with selective binding of an exogenous LGIC ligand. For example, the modified amino acid can confer the modified LGIC with reduced (e.g., minimized or eliminated) binding of an unmodified LGIC subunit (e.g., an LGIC subunit lacking the modification and/or an endogenous LGIC subunit). For example, the modified amino acid can confer the modified LGIC with reduced (e.g., minimized or eliminated) binding of an endogenous LGIC ligand.

Modified LGICs provided herein can be used, for example, in methods for treating channelopathies (e.g., a neural channelopathy or a muscle channelopathy). For example, a modified LGIC, and an exogenous LGIC ligand that can bind to and activate the modified LGIC, can be used to treat a mammal having a channelopathy. In some aspects, a modified LGIC and an exogenous LGIC ligand can be used to modulate (e.g., activate or inhibit) ion transport across the membrane of a cell of a mammal. In some aspects, a modified LGIC and an exogenous LGIC ligand can be used to modulate (e.g., increase or decrease) the excitability of a cell in a mammal.

As used herein a “modified LGIC” is an LGIC that includes at least one “modified LGIC subunit.” Modified LGICs can also be referred to as pharmacologically selective actuator modules (PSAMs). As used herein a “modified LGIC subunit” is an LGIC subunit that includes at least one modified amino acid (e.g., a substituted amino acid) in the LBD and/or at least one modified amino acid (e.g., a substituted amino acid) in the IPD. A modified LGIC subunit described herein can be a modification of an LGIC from any appropriate species (e.g., human, rat, mouse, dog, cat, horse, cow, goat, pig, or monkey). In some aspects, a modified LGIC can include at least one chimeric LGIC subunit having a non-naturally occurring combination of a LBD from a first LGIC and an IPD from a second LGIC.

A modified LGIC (e.g., an LGIC including one or more modified LGIC subunits) can be a homomeric (e.g., having any number of the same modified LGIC subunits) or heteromeric (e.g., having at least one modified LGIC subunit and any number of different LGIC subunits). In some aspects, a modified LGIC described herein can be a homomeric modified LGIC. A modified LGIC described herein can include any suitable number of modified LGIC subunits. In some aspects, a modified LGIC can be a trimer, a tetramer, a pentamer, or a hexamer. For example, a modified LGIC described herein can be a pentamer.

A modified LGIC subunit described herein can include a LBD and/or an IPD from any appropriate LGIC. In some aspects, an LGIC can be a cys-loop LGIC. An LGIC can conduct anions, cations, or both through a cellular membrane in response to the binding of a ligand. For example, the LGIC can transport sodium (Na⁺), potassium (K⁺), calcium (Ca²⁺), and/or chloride (Cl⁻) ions through a cellular membrane in response to the binding of a ligand. Examples of LGICs include, without limitation, AChRs (e.g., nAChRs such as a muscle-type nAChR or a neuronal-type nAChR), gamma-aminobutyric acid (GABA) receptors (e.g., GABA_{A} and GABA_{A}-ρ (also referred to as GABAc)), GlyRs, GluCl receptors, 5HT3 receptors, ionotropic glutamate (iGluR) receptors (e.g., AMPA receptors, kainate receptors, NMDA receptors, and delta receptors), ATP-gated channels (e.g., P2X), and phosphatidylinositol 4,5-bisphosphate (PIP2)-gated channels. In cases where a modified LGIC subunit described herein is a chimeric LGIC subunit, the chimeric LGIC subunit can include a LBD selected from any appropriate LGIC and an IPD selected from any appropriate LGIC. In cases where a LGIC includes multiple different subunits (for example, a neuronal-type nAChR includes α4, β2, and α7 subunits), the LBD and/or IPD can be selected from any of the subunits. For example, a LBD from a nAChR can be a α7 LBD. A representative rat α7 nAChR amino acid sequence (including both a LBD and an IPD) is set forth in SEQ ID NO:4.

In some aspects, a modified LGIC subunit described herein can include a LBD from a α7 nAChR. Examples of α7 nAChR LBDs include, without limitation, a human α7 nAChR LBD having the amino acid sequence set forth in SEQ ID NO: 1, a human α7 nAChR LBD having the amino acid sequence set forth in SEQ ID NO:2, and a human α7 nAChR LBD having the amino acid sequence set forth in SEQ ID NO:3. In some aspects, a α7 nAChR LBD can be a homolog, orthologue, or paralog of the human α7 nAChR LBD set forth in SEQ ID NO:1, SEQ ID NO:2, or SEQ ID NO:3. In some aspects, a α7 nAChR LBD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, or SEQ ID NO:3. Amino acid residues 1-22 of the α7 nAChR LBD set forth in each of SEQ ID NO1, SEQ ID NO:2, and SEQ ID NO:3 contains a signal sequence that can be cleaved from the α7 nAChR LBD. In some aspects, a modified LGIC subunit described herein can include a α7 nAChR LBD having at least 75 percent sequence identity (e.g., at least 80%, at least 82%, at least 85%, at least 88%, at least 90%, at least 93%, at least 95%, at least 97% or at least 99% sequence identity) to residues 23-224 of SEQ ID NO:1, residues 23-229 of SEQ ID NO:2, or residues 23-233 of SEQ ID NO: 11.

In some aspects, a modified LGIC subunit described herein can include a IPD from a 5HT3 receptor. Examples of 5HT3 IPDs include, without limitation, a murine 5HT3 IPD having the amino acid sequence set forth in SEQ ID NO:5, and a human 5HT3 IPD having the amino acid sequence set forth in SEQ ID NO:6. In some aspects, a 5HT3 IPD can be a homolog, orthologue, or paralog of a 5HT3 IPD set forth in SEQ ID NO:5 or SEQ ID NO:6. In some aspects, a 5HT3 IPD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:5 or SEQ ID NO:6.

In calculating percent sequence identity, two sequences are aligned and the number of identical matches of amino acid residues between the two sequences is determined. The number of identical matches is divided by the length of the aligned region (i.e., the number of aligned amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. The alignment of two or more sequences to determine percent sequence identity can be performed using the computer program ClustalW and default parameters, which calculates the best match between a query and one or more subject sequences, and aligns them so that identities, similarities and differences can be determined. See, e.g., Chenna et al., 2003, Nucleic Acids Res., 31 (13): 3497-500.

In cases where a modified LGIC subunit described herein is a chimeric LGIC subunit, the chimeric LGIC subunit can include a LBD and IPD from the same species or a LBD and IPD from different species. In some aspects, a chimeric LGIC subunit can include a LBD from a human LGIC protein and an IPD from a human LGIC protein. For example, a chimeric LGIC subunit can include a human α7 LBD and a human 5HT3 IPD. In some aspects, a chimeric LGIC subunit can include a LBD from a human LGIC protein and an IPD from a murine LGIC protein. For example, a chimeric LGIC subunit can include a human α7 LBD and a murine 5HT3 IPD.

In cases where a modified LGIC subunit described herein is a chimeric LGIC subunit, the chimeric LGIC subunit can include varied fusion points connecting the LBD and the IPD such that the number of amino acids in a LBD may vary when the LBD is fused with different IPDs to form a chimeric channel subunit. For example, the length of an α7 nAChR LBD used to form a chimeric LGIC subunit with a 5HT3 IPD can be different from the length of an α7 nAChR LBD used to form a chimeric LGIC subunit with a GlyR IPD.

A modified LGIC subunit described herein can include a LBD having at least one modified amino acid and/or an IPD having at least one modified amino acid. In some aspects, a modified LGIC subunit described herein can include a LBD having at least one amino acid substitution and an IPD optionally having at least one amino acid substitution. For example, a modified LGIC subunit described herein can include a α7 LBD having at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, SEQ ID NO:3, or SEQ ID NO:4, and can have an amino acid substitution at one or more (e.g., one, two, three, four, five, six, seven, eight, nine, ten, eleven, twelve, or more) of amino acid residues 27, 41, 77, 79, 131, 139, 141, 154, 155, 156, 163, 171, 172, 210, 217, and/or 219. In some aspects, a modified LGIC subunit described herein can include more than one (e.g., two, three, four, five, six, seven, eight, nine, ten, eleven, twelve, or more) amino acid modifications. The modification can be an amino acid substitution. In some aspects, the modified amino acid can confer pharmacological selectivity to the modified LGIC. For example, the modified amino acid can confer the modified LGIC with selective binding of an exogenous LGIC ligand. For example, the modified amino acid can confer the modified LGIC with reduced (minimized or eliminated) binding of an unmodified LGIC subunit (an LGIC subunit lacking the modification and/or an endogenous LGIC subunit). For example, the modified amino acid can confer the modified LGIC with reduced (minimized or eliminated) binding of an endogenous LGIC ligand.

When a modified LGIC subunit included in a modified LGIC provided herein includes a modified amino acid (e.g., an amino acid substitution) at residue 154 of a LBD (e.g., a α7 LBD), the modified amino acid can be a substitution of the Val at residue 154 with any other amino acid. Examples of amino acids that can substituted for the Val at residue 154 of a LBD (e.g., as numbered in SEQ ID NO:1, SEQ ID NO:2, SEQ ID NO:3, and SEQ ID NO: 4) can include, without limitation, Ile and Leu. For example, a modified LGIC subunit included in a modified LGIC provided herein can include a LBD (e.g., a α7 LBD) having at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, SEQ ID NO:3, or SEQ ID NO:4, provided that it also has a modified amino acid (e.g., an amino acid substitution) at residue 154. For example, a modified LGIC subunit included in a modified LGIC provided herein can include a LBD (e.g., a α7 LBD) having one or more amino acid substitutions within the articulated sequence of the sequence identifier (e.g., SEQ ID NO:1, SEQ ID NO:2, SEQ ID NO:3, or SEQ ID NO:4), can have one or more amino acid residues preceding the articulated sequence of the sequence identifier (e.g., SEQ ID NO:1, SEQ ID NO:2, SEQ ID NO:3, or SEQ ID NO: 4), and/or can have one or more amino acid residues following the articulated sequence of the sequence identifier (e.g., SEQ ID NO:1, SEQ ID NO:2, SEQ ID NO:3, or SEQ ID NO: 4), provided that it also has a modified amino acid (e.g., an amino acid substitution) at residue 154.

When a modified LGIC subunit included in a modified LGIC provided herein includes a modified amino acid (e.g., an amino acid substitution) at residue 155 of a LBD (e.g., a α7 LBD), the modified amino acid can be a substitution of the Arg at residue 155 with any other amino acid. Examples of amino acids that can substituted for the Arg at residue 155 of a LBD (e.g., as numbered in SEQ ID NO:1, SEQ ID NO:2, SEQ ID NO:3, and SEQ ID NO: 4) can include, without limitation, Tyr. For example, a modified LGIC subunit included in a modified LGIC provided herein can include a LBD (e.g., a α7 LBD) having at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, SEQ ID NO:3, or SEQ ID NO:4, provided that it also has a modified amino acid (e.g., an amino acid substitution) at residue 155. For example, a modified LGIC subunit included in a modified LGIC provided herein can include a LBD (e.g., a α7 LBD) having one or more amino acid substitutions within the articulated sequence of the sequence identifier (e.g., SEQ ID NO:1, SEQ ID NO:2, SEQ ID NO:3, or SEQ ID NO: 4), can have one or more amino acid residues preceding the articulated sequence of the sequence identifier (e.g., SEQ ID NO:1, SEQ ID NO:2, SEQ ID NO:3, or SEQ ID NO:4), and/or can have one or more amino acid residues following the articulated sequence of the sequence identifier (e.g., SEQ ID NO:1, SEQ ID NO:2, SEQ ID NO:3, or SEQ ID NO:4), provided that it also has a modified amino acid (e.g., an amino acid substitution) at residue 155.

When a modified LGIC subunit included in a modified LGIC provided herein includes a modified amino acid (e.g., an amino acid substitution) at residue 156 of a LBD (e.g., a α7 LBD), the modified amino acid can be a substitution of the Trp at residue 156 with any other amino acid. Examples of amino acids that can substituted for the Trp at residue 156 of a LBD (e.g., as numbered in SEQ ID NO:1, SEQ ID NO:2, SEQ ID NO:3, and SEQ ID NO: 4) can include, without limitation, Asn and Gln. For example, a modified LGIC subunit included in a modified LGIC provided herein can include a LBD (e.g., a α7 LBD) having at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, SEQ ID NO:3, or SEQ ID NO:4, provided that it also has a modified amino acid (e.g., an amino acid substitution) at residue 156. For example, a modified LGIC subunit included in a modified LGIC provided herein can include a LBD (e.g., a α7 LBD) having one or more amino acid substitutions within the articulated sequence of the sequence identifier (e.g., SEQ ID NO:1, SEQ ID NO:2, SEQ ID NO:3, or SEQ ID NO:4), can have one or more amino acid residues preceding the articulated sequence of the sequence identifier (e.g., SEQ ID NO:1, SEQ ID NO:2, SEQ ID NO:3, or SEQ ID NO: 4), and/or can have one or more amino acid residues following the articulated sequence of the sequence identifier (e.g., SEQ ID NO:1, SEQ ID NO:2, SEQ ID NO:3, or SEQ ID NO: 4), provided that it also has a modified amino acid (e.g., an amino acid substitution) at residue 156.

When a modified LGIC subunit included in a modified LGIC provided herein includes a modified amino acid (e.g., an amino acid substitution) at residue 163 of a LBD (e.g., a α7 LBD), the modified amino acid can be a substitution of the His at residue 163 with any other amino acid. Examples of amino acids that can substituted for the His at residue 163 of a LBD (e.g., as numbered in SEQ ID NO:1, SEQ ID NO:2, SEQ ID NO:3, and SEQ ID NO: 4) can include, without limitation, Thr, Ala, Asp, Phe, Gly, Lys, Asn, Arg, Ser, and Val. For example, a modified LGIC subunit included in a modified LGIC provided herein can include a LBD (e.g., a α7 LBD) having at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, SEQ ID NO:3, or SEQ ID NO:4, provided that it also has a modified amino acid (e.g., an amino acid substitution) at residue 163. For example, a modified LGIC subunit included in a modified LGIC provided herein can include a LBD (e.g., a α7 LBD) having one or more amino acid substitutions within the articulated sequence of the sequence identifier (e.g., SEQ ID NO:1, SEQ ID NO:2, SEQ ID NO:3, or SEQ ID NO:4), can have one or more amino acid residues preceding the articulated sequence of the sequence identifier (e.g., SEQ ID NO:1, SEQ ID NO: 2, SEQ ID NO:3, or SEQ ID NO:4), and/or can have one or more amino acid residues following the articulated sequence of the sequence identifier (e.g., SEQ ID NO:1, SEQ ID NO: 2, SEQ ID NO:3, or SEQ ID NO:4), provided that it also has a modified amino acid (e.g., an amino acid substitution) at residue 163.

When a modified LGIC subunit included in a modified LGIC provided herein includes a modified amino acid (e.g., an amino acid substitution) at residue 172 of a LBD (e.g., a α7 LBD), the modified amino acid can be a substitution of the Ser at residue 172 with any other amino acid. Examples of amino acids that can substituted for the Ser at residue 172 of a LBD (e.g., as numbered in SEQ ID NO:1, SEQ ID NO:2, SEQ ID NO:3, and SEQ ID NO: 4) can include, without limitation, Ala, Val, and Ile. For example, a modified LGIC subunit included in a modified LGIC provided herein can include a LBD (e.g., a α7 LBD) having at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, SEQ ID NO:3, or SEQ ID NO:4, provided that it also has a modified amino acid (e.g., an amino acid substitution) at residue 172. For example, a modified LGIC subunit included in a modified LGIC provided herein can include a LBD (e.g., a α7 LBD) having one or more amino acid substitutions within the articulated sequence of the sequence identifier (e.g., SEQ ID NO:1, SEQ ID NO:2, SEQ ID NO:3, or SEQ ID NO:4), can have one or more amino acid residues preceding the articulated sequence of the sequence identifier (e.g., SEQ ID NO:1, SEQ ID NO:2, SEQ ID NO:3, or SEQ ID NO: 4), and/or can have one or more amino acid residues following the articulated sequence of the sequence identifier (e.g., SEQ ID NO:1, SEQ ID NO:2, SEQ ID NO:3, or SEQ ID NO: 4), provided that it also has a modified amino acid (e.g., an amino acid substitution) at residue 172.

In some aspects, a modified LGIC subunit described herein can include at least one modified amino acid that confers the modified LGIC with selective binding (e.g., enhanced binding or increased potency) with an exogenous LGIC ligand. The binding with an exogenous LGIC ligand can be selective over the binding with an endogenous LGIC ligand. A modified LGIC subunit with selective binding with an exogenous LGIC ligand can include any appropriate LDB (e.g., a α7 LBD). In some aspects, the modified LGIC subunit can include a α7 LBD set forth in SEQ ID NO:1, SEQ ID NO:2, SEQ ID NO:3, or SEQ ID NO:4, and the amino acid modification can be a substitution at amino acid residue 77, 79, 131, 139, 141, 154, 155, 156, 163, 171, 172, and/or 217. In some aspects, the tryptophan at amino acid residue 77 of SEQ ID NO:1, SEQ ID NO:2, SEQ ID NO:3, or SEQ ID NO:4 can be substituted with a hydrophobic amino acid residue such as phenylalanine (e.g., W77F), tyrosine (e.g., W77Y), or methionine (e.g., W77M). For example, a modified LGIC subunit described herein can include a α7 LBD set forth in SEQ ID NO:1, SEQ ID NO:2, SEQ ID NO: 3, or SEQ ID NO:4 and having a W77F substitution. In some aspects, the glutamine at amino acid residue 79 of SEQ ID NO:1, SEQ ID NO:2, SEQ ID NO:3, or SEQ ID NO:4 can be substituted with an amino acid residue such as alanine (e.g., Q79A), glycine (e.g., Q79G), or serine (e.g., Q79S). For example, a modified LGIC subunit described herein can include a α7 LBD having a Q79G substitution. In some aspects, the leucine at amino acid residue 131 of SEQ ID NO:1, SEQ ID NO:2, SEQ ID NO:3, or SEQ ID NO:4 can be substituted with an amino acid residue such as alanine (e.g., L131A), glycine (e.g., L131G), methionine (e.g., L131M), asparagine (e.g., L131N), glutamine (e.g., L131Q), valine (e.g., L131V), or phenylalanine (e.g., L131F). In some aspects, the glutamine at amino acid residue 131 of SEQ ID NO:1, SEQ ID NO:2, SEQ ID NO:3, or SEQ ID NO:4 can be substituted with an amino acid residue such as glycine (e.g., Q139G) or leucine (e.g., Q139L). For example, a modified LGIC subunit described herein can include a α7 LBD set forth in SEQ ID NO:1, SEQ ID NO:2, SEQ ID NO:3, or SEQ ID NO:4 and having a Q139L substitution. In some aspects, the valine at amino acid residue 154 of SEQ ID NO:1, SEQ ID NO:2, SEQ ID NO:3, or SEQ ID NO:4 can be substituted with an amino acid residue such as isoleucine (e.g., V154I) or leucine (e.g., V154L). For example, a modified LGIC subunit described herein can include a α7 LBD set forth in SEQ ID NO:1, SEQ ID NO:2, SEQ ID NO:3, or SEQ ID NO: 4 and having a V154I substitution. In some aspects, the arginine at amino acid residue 155 of SEQ ID NO:1, SEQ ID NO:2, SEQ ID NO:3, or SEQ ID NO:4 can be substituted with an amino acid residue such as tyrosine (e.g., R155Y). For example, a modified LGIC subunit described herein can include a α7 LBD set forth in SEQ ID NO:1, SEQ ID NO:2, SEQ ID NO:3, or SEQ ID NO:4 and having a R155Y substitution. In some aspects, the tryptophan at amino acid residue 156 of SEQ ID NO:1, SEQ ID NO:2, SEQ ID NO:3, or SEQ ID NO:4 can be substituted with an amino acid residue such as glutamine (e.g., W156Q) or asparagine (e.g., W156N). For example, a modified LGIC subunit described herein can include a α7 LBD set forth in SEQ ID NO:1, SEQ ID NO:2, SEQ ID NO:3, or SEQ ID NO:4 and having a W156N substitution. In some aspects, the histidine at amino acid residue 163 of SEQ ID NO:1, SEQ ID NO:2, SEQ ID NO:3, or SEQ ID NO:4 can be substituted with an amino acid residue such as threonine (e.g., H163T), alanine (e.g., H163A), aspartic acid (e.g., H163D), phenylalanine (e.g., H162F), glycine (e.g., H163G), lysine (e.g., H163K), asparagine (e.g., H163N), arginine (e.g., H163R), serine (e.g., H163S), or valine (e.g., H163V). For example, a modified LGIC subunit described herein can include a α7 LBD set forth in SEQ ID NO:1, SEQ ID NO:2, SEQ ID NO:3, or SEQ ID NO:4 and having a H163T substitution. In some aspects, the serine at amino acid residue 172 of SEQ ID NO: 1, SEQ ID NO:2, SEQ ID NO:3, or SEQ ID NO:4 can be substituted with an amino acid residue such as alanine (e.g., S172A), valine (e.g., S172V), or isoleucine (e.g., S172I). For example, a modified LGIC subunit described herein can include a α7 LBD set forth in SEQ ID NO:1, SEQ ID NO:2, SEQ ID NO:3, or SEQ ID NO:4 and having a S172V or S172A substitution. In some aspects, the tyrosine at amino acid residue 217 of SEQ ID NO: 1, SEQ ID NO:2, SEQ ID NO:3, or SEQ ID NO:4 can be substituted with an amino acid residue such as phenylalanine (e.g., Y217F). For example, a modified LGIC subunit described herein can include a α7 LBD set forth in SEQ ID NO:1, SEQ ID NO:2, SEQ ID NO: 3, or SEQ ID NO:4 and having a Y217F substitution.

A modified LGIC subunit with selective binding with an exogenous LGIC ligand can include any appropriate IPD (e.g., a GlyR IPD, a GABA_{A}-ρ IPD, or a 5HT3 IPD). In some aspects, a modified LGIC subunit can include a 5HT3 IPD. For example, a modified LGIC subunit included in a modified LGIC provided herein can include an IPD (e.g., a 5HT3 IPD) having at least 75 percent sequence identity (e.g., at least 80%, at least 82%, at least 85%, at least 88%, at least 90%, at least 93%, at least 95%, at least 97% or at least 99% sequence identity) to SEQ ID NO:5 or SEQ ID NO:6. For example, a modified LGIC subunit included in a modified LGIC provided herein can include an IPD (e.g., a 5HT3 IPD) having one or more amino acid substitutions within the articulated sequence of the sequence identifier (e.g., SEQ ID NO:5 or SEQ ID NO:6), can have one or more amino acid residues preceding the articulated sequence of the sequence identifier (e.g., SEQ ID NO:5 or SEQ ID NO:6), and/or can have one or more amino acid residues following the articulated sequence of the sequence identifier (e.g., SEQ ID NO:5 or SEQ ID NO:6).

When a modified LGIC subunit described herein includes an IPD having at least one modified amino acid (e.g., at least one amino acid substitution), the IPD can include any appropriate amino acid modifications. In some aspects, a modified LGIC subunit described herein can include an IPD (e.g., a 5HT3 IPD) having at least 75 percent sequence identity (e.g., at least 80%, at least 82%, at least 85%, at least 88%, at least 90%, at least 93%, at least 95%, at least 97% or at least 99% sequence identity) to SEQ ID NO:5, and can have an amino acid substitution at one or more (e.g., one, two, or three) of amino acid residues 425, 429, and/or 433. For example, an IPD (e.g., a 5HT3 IPD) can include a R425Q substitution, a R429D substitution, and/or a R433A. In some aspects, a modified LGIC subunit described herein can include an IPD (e.g., a 5HT3 IPD) having at least 75 percent sequence identity (e.g., at least 80%, at least 82%, at least 85%, at least 88%, at least 90%, at least 93%, at least 95%, at least 97% or at least 99% sequence identity) to SEQ ID NO:6, and can have an amino acid substitution at one or more (e.g., one, two, or three) of amino acid residues 420, 424, and/or 428. For example, an IPD (e.g., a 5HT3 IPD) can include a R420Q substitution, a R424D substitution, and/or a R428A substitution.

Additional examples of modifications that can confer the modified LGIC with selective binding of an exogenous LGIC ligand include modifications described elsewhere (see, e.g., U.S. Pat. No. 8,435,762, WO 2018/009832, and WO 2019/094778).

A modified LGIC subunit that selectively binds (e.g., enhanced binding or increased potency) an exogenous LGIC ligand over an endogenous (e.g., a canonical) LGIC ligand can also be described as having enhanced potency for an exogenous ligand. In some aspects, a modified LGIC subunit described herein that selectively binds an exogenous LGIC ligand can have at least 4 fold (e.g., at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, or at least 20 fold) enhanced potency for an exogenous ligand. In some aspects, a modified LGIC subunit described herein that selectively binds an exogenous LGIC ligand can have about 4 fold to about 200 fold (e.g., about 4 fold to about 200 fold, about 5 fold to about 180 fold, about 6 fold to about 175 fold, about 7 fold to about 150 fold, about 8 fold to about 125 fold, about 9 fold to about 100 fold, about 10 fold to about 90 fold, about 11 fold to about 75 fold, about 12 fold to about 65 fold, about 13 fold to about 50 fold, about 14 fold to about 40 fold, or about 15 fold to about 30 fold) enhanced potency for an exogenous ligand. For example, a modified LGIC subunit described herein that selectively binds an exogenous LGIC ligand can have about 10 fold to about 100 fold enhanced potency for an exogenous ligand. For example, a modified LGIC subunit described herein that selectively binds an exogenous LGIC ligand can have about 10 fold to about 20 fold enhanced potency for an exogenous ligand.

In some aspects, a modified LGIC described herein can include at least one chimeric α7-5HT3 LGIC subunit having an α7 nAChR LBD having a substitution at amino acid residue 154 (e.g., V154I or V154L), and a 5HT3 IPD. For example, a modified LGIC subunit can include an α7 nAChR LBD having at least 75% sequence identity to SEQ ID NO: 1, SEQ ID NO:2, SEQ ID NO:3, or SEQ ID NO:4 and a substitution at amino acid residue 154 (e.g., V154I or V154L), and can include a 5HT3 IPD having at least 75% sequence identity to SEQ ID NO:5 or SEQ ID NO:6.

In some aspects, a modified LGIC described herein can include at least one chimeric α7-5HT3 LGIC subunit having an α7 nAChR LBD having a substitution at amino acid residue 155 (e.g., R155Y), and a 5HT3 IPD. For example, a modified LGIC subunit can include α7-5HT3 LGIC subunit having an α7 nAChR LBD having at least 75% sequence identity to SEQ ID NO:1, SEQ ID NO:2, SEQ ID NO:3, or SEQ ID NO:4 and a substitution at amino acid residue 155 (e.g., R155Y), and a 5HT3 IPD having at least 75% sequence identity to SEQ ID NO:5 or SEQ ID NO:6.

In some aspects, a modified LGIC described herein can include at least one chimeric α7-5HT3 LGIC subunit having an α7 nAChR LBD having a substitution at amino acid residue 156 (e.g., W156Q or W156N), and a 5HT3 IPD. For example, a modified LGIC subunit described herein can include at least one chimeric α7-5HT3 LGIC subunit having an α7 nAChR LBD having at least 75% sequence identity to SEQ ID NO:1, SEQ ID NO:2, SEQ ID NO: 3, or SEQ ID NO:4 and a substitution at amino acid residue 156 (e.g., W156Q or W156N), and a 5HT3 IPD having at least 75% sequence identity to SEQ ID NO:5 or SEQ ID NO: 6.

In some aspects, a modified LGIC described herein can include at least one chimeric α7-5HT3 LGIC subunit having an α7 nAChR LBD having a substitution at amino acid residue 163 (e.g., H163T, H163A, H163D, H162F, H163G, H163K, H163N, H163R, H163S, or H163V), and a 5HT3 IPD. For example, a modified LGIC subunit can include an α7 nAChR LBD having at least 75% sequence identity to SEQ ID NO:1, SEQ ID NO:2, SEQ ID NO: 3, or SEQ ID NO:4 and a substitution at amino acid residue 163 (e.g., H163T, H163A, H163D, H162F, H163G, H163K, H163N, H163R, H163S, or H163V), and a 5HT3 IPD having at least 75% sequence identity to SEQ ID NO:5 or SEQ ID NO:6.

In some aspects, a modified LGIC described herein can include at least one chimeric α7-5HT3 LGIC subunit having an α7 nAChR LBD having a substitution at amino acid residue 172 (e.g., S172A, S172V, or S172I), and a 5HT3 IPD. For example, a modified LGIC subunit can include an α7 nAChR LBD having at least 75% sequence identity to SEQ ID NO: 1, SEQ ID NO:2, SEQ ID NO:3, or SEQ ID NO:4 and a substitution at amino acid residue 172 (e.g., S172A, S172V, or S172I), and a 5HT3 IPD having at least 75% sequence identity to SEQ ID NO:5 or SEQ ID NO:6. In some aspects, a modified LGIC described herein can include one or more additional polypeptide sequences. A polypeptide sequence can be a transport sequence (e.g., an export sequence and/or a signal sequence). Examples of export sequences include, without limitation, ER export sequences (e.g., FCYENEV (SEQ ID NO: 9)). Examples of signal sequences include, without limitation, CHRNB4 signal sequences (e.g., MRRAPSLVLFFLVALCGRGNC (SEQ ID NO:10)). A polypeptide sequence can be a targeting sequence. Examples of targeting sequences include, without limitation, KCNB1 somatic targeting sequences (e.g., QSQPILNTKEMAPQSKPPEELEMSSMPSPVAPLPARTEGVIDMRSMSSIDSFISCATDFP EATRF (SEQ ID NO:11)). The one or more additional polypeptide sequences can be included in a modified LGIC in any appropriate location. In some aspects, an additional polypeptide sequence can be a terminal (e.g., a C-terminal or an N-terminal) polypeptide sequence. In some aspects, an additional polypeptide sequence can be an insertion. In some aspects, an additional polypeptide sequence can be a substitution. One or more additional polypeptide sequences, when present, can be on any one or more of the modified LGIC subunits present in a modified LGIC described herein.

In some aspects, a modified LGIC described herein can include an export sequence. For example, a modified LGIC can include an ER export sequence (e.g., SEQ ID NO:9). An export sequence, when present, can be on any one or more of the modified LGIC subunits present in a modified LGIC described herein.

In some aspects, a modified LGIC described herein can include a signal sequence. For example, a modified LGIC can include a CHRNB4 signal sequence (e.g., SEQ ID NO: 10). A signal sequence, when present, can be on any one or more of the modified LGIC subunits present in a modified LGIC described herein.

In some aspects, a modified LGIC described herein can include a targeting sequence. For example, a modified LGIC can include a KCNB1 somatic targeting sequence (e.g., SEQ ID NO: 11). A targeting sequence, when present, can be on any one or more of the modified LGIC subunits present in a modified LGIC described herein.

In cases where a LBD and/or a IPD is a homolog, orthologue, or paralog of a sequence set forth herein (e.g., SEQ ID NOs: 1-5 and/or 9), it is understood that reference to a particular modified amino acid residue can shift to the corresponding amino acid in the homolog, orthologue, or paralog. For example, residues 425, 429, and 433 in a murine 5HT3 IPD set forth in SEQ ID NO:5 correspond to residues 420, 424, and 428 in a human 5HT3 IPD set forth in SEQ ID NO:6, and the R425Q, R429D, and R433A substitutions in a murine 5HT3 IPD correspond to R420Q, R424D, and R428A substitutions in a human 5HT3 IPD. A targeting sequence, when present, can be on any one or more of the modified LGIC subunits present in a modified LGIC described herein.

Any method can be used to obtain a modified LGIC subunit described herein. In some aspects, peptide synthesis methods can be used to make a modified LGIC subunit described herein. Examples of methods of peptide synthesis include, without limitation, liquid-phase peptide synthesis, and solid-phase peptide synthesis. In some aspects, protein biosynthesis methods can be used to make a modified LGIC subunit described herein. Examples of methods of protein biosynthesis include, without limitation, transcription and/or translation of nucleic acids encoding a phosphorylation-mimicking peptide provided herein. Similar modified LGIC subunits (e.g., modified subunits having essentially the same modifications and/or having essentially the same amino acid sequence) will self-assemble through interactions between the LBDs to form a modified LGIC.

This document also provides nucleic acids encoding modified LGIC subunits described herein as well as constructs (e.g., synthetic constructs such as plasmids, non-viral vectors, viral vectors (such as adeno-associated virus, a herpes simplex virus, or lentivirus vectors)) for expressing nucleic acids encoding modified LGIC subunits described herein.

A nucleic acid sequence encoding modified LGIC subunit described herein can encode any LGIC described herein. In some aspects, a nucleic acid sequence provided herein can encode a LBD from any LGIC described herein. In some aspects, a nucleic acid sequence provided herein can encode an IPD from any LGIC described herein. In cases where a nucleic acid sequence provided herein encodes a chimeric LGIC subunit, the chimeric LGIC subunit can include a LBD selected from any appropriate LGIC and an IPD selected from any appropriate LGIC.

In some aspects, a nucleic acid sequence can encode a LGIC described herein. For example, a nucleic acid sequence can encode a nAChR (e.g., a α7 nAChR). A representative nucleic acid sequence encoding a rat α7 nAChR amino acid sequence (including both a LBD and an IPD) is set forth in SEQ ID NO:12.

In some aspects, a nucleic acid sequence encoding a modified LGIC subunit described herein can encode a LBD from a α7 nAChR. Examples of nucleic acid sequences encoding α7 nAChR LBDs include, without limitation, a nucleic acid sequence set forth in SEQ ID NO:13, a nucleic acid sequence set forth in SEQ ID NO:14, and a nucleic acid sequence set forth in SEQ ID NO:15. In some aspects, a nucleic acid sequence encoding α7 nAChR LBD can have at least 75 percent sequence identity (e.g., at least 80%, at least 82%, at least 85%, at least 88%, at least 90%, at least 93%, at least 95%, at least 97% or at least 99% sequence identity) to SEQ ID NO: 13, SEQ ID NO:14, or SEQ ID NO:15.

In some aspects, a nucleic acid sequence encoding a modified LGIC subunit described herein can encode an IPD from a 5HT3 receptor. Examples of nucleic acid sequences encoding 5HT3 IPDs include, without limitation, a nucleic acid sequence set forth in SEQ ID NO: 16, and a nucleic acid sequence set forth in SEQ ID NO: 17. In some aspects, a nucleic acid sequence encoding a 5HT3 IPD can have at least 75 percent sequence identity (e.g., at least 80%, at least 82%, at least 85%, at least 88%, at least 90%, at least 93%, at least 95%, at least 97% or at least 99% sequence identity) to SEQ ID NO:16 or SEQ ID NO:17.

In calculating percent sequence identity, two sequences are aligned and the number of identical matches of amino acid residues between the two sequences is determined. The number of identical matches is divided by the length of the aligned region (i.e., the number of aligned nucleic acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. The alignment of two or more sequences to determine percent sequence identity can be performed using the computer program ClustalW and default parameters, which calculates the best match between a query and one or more subject sequences, and aligns them so that identities, similarities and differences can be determined. See, e.g., Chenna et al., 2003, Nucleic Acids Res., 31 (13): 3497-500.

A nucleic acid sequence encoding a modified LGIC described herein can include at least one modified nucleic acid such that the nucleic acid sequence can encode a LBD having at least one modified amino acid and/or an IPD having at least one modified amino acid. In some aspects, a nucleic acid sequence encoding a modified LGIC described herein can include more than one (e.g., two, three, four, five, six, seven, eight, nine, ten, eleven, twelve, or more) modified nucleic acids. For example, a nucleic acid sequence can encode a modified LGIC subunit including a α7 LBD having at least 75 percent sequence identity to SEQ ID NO:1, SEQ ID NO:2, SEQ ID NO:3, or SEQ ID NO:4, and an amino acid substitution at amino acid residue 27, 41, 77, 79, 131, 139, 141, 154, 155, 156, 163, 171, 172, 210, 217, and/or 219. Examples of nucleic acid codons at codon numbers 27, 41, 77, 79, 131, 139, 141, 154, 155, 156, 163, 171, 172, 210, 217, and/or 219 that can results amino acid substitutions at amino acid residues 27, 41, 77, 79, 131, 139, 141, 154, 155, 156, 163, 171, 172, 210, 217, and/or 219 can be as shown below.

TABLE 1
Codon usage resulting in LBD amino acid substitutions

| WT amino | |
|---|---|
| acid | |
| residue | Modified amino acid: Modified nucleic acid codon |
| R27 | D: GAC |
| E41 | R: AGG |
| W77 | F: TTC, Y: TAC |
| Q79 | A: GCA, G: GGC, S: TCG |
| Y115 | F: TTC |
| L131 | A: GCG, F: TTC, G: GGA, I: ATA, M: ATG, N: AAC, Q: |
| | CAA, V: GTG |
| Q139 | G: GGC, L: CTG |
| L141 | F: TTC or TTT |
| V154 | I: ATC, L: CTC |
| R155 | Y: TAC |
| W156 | N: AAC, Q: CAG |
| H163 | A: GCA, D: GAC, F: TTT, G: GGA, K: AAA, N: AAC, R: |
| | CGA, S: TCA, T: ACC, V: GTA |
| W171 | F: TTT, Y: TAC |
| S172 | A: GCA, I: ATA, V: GTA |
| Y210 | F: TTT |
| Y217 | A: GCC, F: TTC, F: TTT, H: CAT, V: GTA |
| D219 | A: GCT |

In some aspects, a nucleic acid sequence encoding a modified LGIC subunit described herein can encode a α7-5HT3 chimeric LGIC subunit as set forth in SEQ ID NO:7 (e.g., including a human α7 nAChR LBD (SEQ ID NO:1) and a murine 5HT3 IPD (SEQ ID NO: 5) components). Examples of nucleic acid sequences encoding a α7-5HT3 chimeric LGIC subunit including a human α7 nAChR LBD and a murine 5HT3 IPD include, without limitation, a nucleic acid sequence set forth in SEQ ID NO: 18.

In some aspects, a nucleic acid sequence encoding a modified LGIC described herein can include a nucleic acid sequence encoding one or more additional polypeptide sequences (e.g., a transport sequence such as an export sequence and/or a signal sequence, or a targeting sequence). Examples of nucleic acid sequences encoding export sequences include, without limitation, a nucleic acid sequence encoding an ER export sequence (e.g., SEQ ID NO:19). Examples of nucleic acid sequences encoding signal sequences include, without limitation, a nucleic acid sequence encoding a CHRNB4 signal sequence (e.g., SEQ ID NO:20). Examples of nucleic acid sequences encoding targeting sequences include, without limitation, a nucleic acid sequence encoding a KCNB1 somatic targeting sequence (e.g., SEQ ID NO: 21).

In some aspects, a nucleic acid encoding a modified LGIC subunit described herein can be linked (e.g., operably linked) to one or more regulatory elements. For example, nucleic acids encoding modified LGIC subunits described herein can be operably linked to any appropriate promoter. A promoter can be a native (i.e., minimal) promoter or a composite promoter. A promoter can be a ubiquitous (i.e., constitutive) promoter or a regulated promoter (e.g., inducible, tissue specific, cell-type specific (e.g., neuron specific, muscle specific, glial specific), and neural subtype-specific). Examples of promoters that can be used to drive expression of nucleic acids encoding modified LGIC subunits described herein include, without limitation, synapsin (SYN), CAMKII, CMV, CAG, enolase, TRPVI, POMC, NPY, AGRP, MCH, and Orexin promoters. In some aspects, a nucleic acid encoding a modified LGIC subunit described herein can be operably linked to a neuron specific promoter.

In cases where nucleic acids encoding modified LGIC subunits described herein are present in a construct, the construct can be any appropriate construct. A construct can be a nucleic acid (e.g., DNA, RNA, or a combination thereof) construct. Examples of constructs include, without limitation, plasmids, non-viral vectors, viral vectors (e.g., adeno-associated virus (AAV) vectors, herpes simplex virus vectors, or lentivirus vectors).

The term “adeno-associated virus vector” or “AAV vector” as used herein refers to any vector that comprises or derives from components of an adeno-associated virus vector and is suitable to infect mammalian cells, e.g., human cells. The term “AAV vector” typically designates an AAV-type viral particle or virion or an AAV genome comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, capsid serotypes AAV type 1 (GenBank Accession Number NP_049542.1), AAV type 2 (GenBank Accession Number YP_680426 (VP1), YP_680427 (VP2), and YP_680428 (VP3)), AAV type 3 (including types 3A and 3B) (GenBank Accession Numbers NP_043941.1 (3 A) and NP_045760.1 (3B)), AAV type 4 (GenBank Accession Number NP_044927.1), AAV type 5 (GenBank Accession Number YP_068409), AAV type 6 (GenBank Accession Number NP_045758.1), AAV type 7 (GenBank Accession Number YP_077178), AAV type 8 (GenBank Accession Numbers YP_077180), AAV type 9 (GenBank Accession Number AY530579), AAV type 10, AAV type 11, AAV type 12, AAV type 13, AAVrh8 (GenBank Accession Number AY242997), AAVrhlO (GenBank Accession Number AY243015), AAVrh. 74, snake AAV (GenBank Accession Number YP_068094), avian AAV (GenBank Accession Numbers NP_852781 (VR-865) and YP_077183 (DA-1)), bovine AAV (GenBank Accession Number YP_024971 (capsid)), canine AAV, equine AAV, ovine AAV, goat AAV, shrimp AAV, the AAV serotypes and clades disclosed by Gao et al. (J. Virol. 78:6381 (2004)) and Moris et al. (Virol. 33:375 (2004)), and any other AAV. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably.

In some aspects, the nucleic acids (e.g., a modified nucleic acid), polynucleotides, or expression constructs disclosed herein can be administered as a component of a packaged viral vector. In general, packaged viral vectors include a viral vector packaged in a capsid.

In some aspects, the viral vector is an AAV vector. In some aspects, an AAV vector as used herein can comprise a recombinant AAV vector (rAAV). A “rAAV vector” as used herein refers to a recombinant vector comprising part of an AAV genome packaged in a protein shell of capsid (Cap) protein derived from an AAV serotype as disclosed herein. Part of an AAV genome can contain the inverted terminal repeats (ITR) derived from an adeno-associated virus serotype, such as AAV1, AAV2, AAV3, AAV4, AAV5, AAV6, AAV7, AAV8, AAVrh8, AAV9, AAV10, AAVRrhlO, AAV11, AAV 12, and others (see, e.g., Earley et al., Hum. Gene Ther. 31:151-162, 2020, incorporated herein by reference in its entirety). In some aspects, the ITR is derived from AAV5.

The complete genome of several AAV serotypes and corresponding ITR has been sequenced (see, e.g., Chiorini et al. 1999, J. of Virology, 73 (2): 1309-19). They can be either cloned or made by chemical synthesis as known in the art, using for example an oligonucleotide synthesizer as supplied e.g. by Applied Biosystems Inc. (Fosters, Calif., USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the wild type AAV sequence between the ITRs can be replaced with the desired nucleotide sequence.

In some aspects, the AAV capsid and/or AAV vector is of an AAV1, AAV2, AAV3, AAV4, AAV5, AAV6, AAV7, AAV8, AAVrh8, AAV9, AAVrh9, AAV10, AAVrhlO, AAV11, or AAV12 serotype. In some aspects, the AAV capsid and/or AAV vector is an AAV5 serotype.

In some aspects, a construct including a nucleic acid encoding a modified LGIC subunit described herein can express the modified LGIC subunit. In some aspects, a construct can include an internal ribosome entry site (IRES; e.g., a bicistronic IRES). In some aspects, a construct can include a nucleic acid sequence encoding a detectable marker (e.g., a fluorescent polypeptide such as a green fluorescent polypeptide (GFP; e.g., an enhanced GFP (EGFP))). In some aspects, a construct can include a nucleic acid sequence providing the construct with a selectable marker (e.g., an antibiotic resistance marker such as ampicillin resistance).

This document also provides cells (e.g., mammalian cells) having a modified LGIC described herein. Mammalian cells having a modified LGIC described herein can be obtained by any appropriate method. In some aspects, a pre-assembled modified LGIC can be provided to the cell. In some aspects, a nucleic acid encoding a modified LGIC subunit described herein can be provided to the cell under conditions in which a modified LGIC subunit is translated and under conditions in which multiple (e.g., three, four, five, six, or more) modified LGIC subunits can assemble into a modified LGIC described herein.

In some aspects, this document features an isolated cell in culture comprising a nucleic acid encoding a modified ligand gated ion channel (LGIC) comprising at least one modified LGIC subunit, the modified LGIC subunit comprising: (a) a modified alpha7 nicotinic acetylcholine receptor (a7-nAChR) ligand binding domain (LBD), wherein the modified a7-nAChR LBD comprises: (i) an amino acid sequence having at least 93% sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) an IPD.

In some aspects, this document features an isolated cell in culture comprising a nucleic acid encoding a modified ligand gated ion channel (LGIC) comprising at least one modified LGIC subunit, the modified LGIC subunit comprising: (a) a modified alpha7 nicotinic acetylcholine receptor (a7-nAChR) ligand binding domain (LBD), wherein the modified a7-nAChR LBD comprises: (i) an amino acid sequence having at least 93% sequence identity to a sequence set forth in SEQ ID NO: 1, SEQ ID NO:2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and (b) a serotonin 3 receptor (5HT3) ion pore domain (IPD).

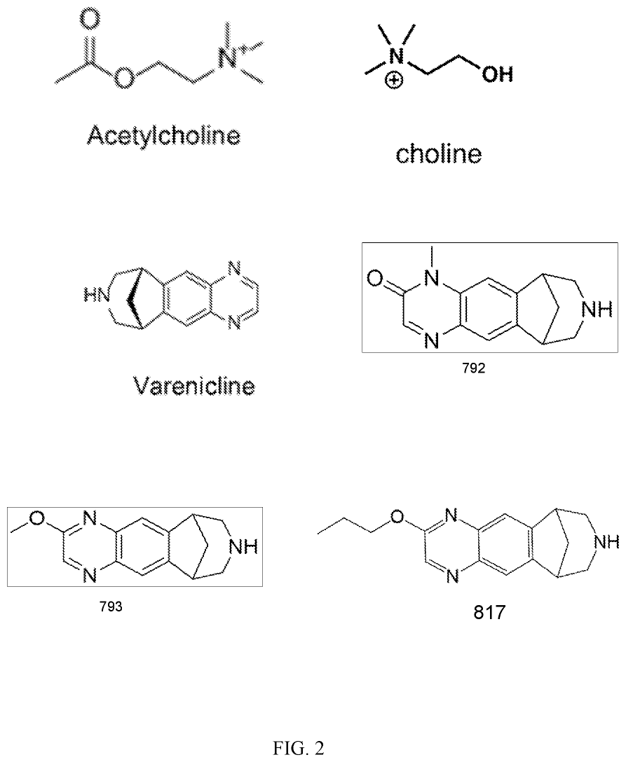

Modified LGICs described herein can be activated by any appropriate LGIC ligand. LGIC ligands can also be referred to as pharmacologically selective effector molecules (PSEMs). A LGIC ligand that can bind to and activate modified LGICs described herein can be exogenous or endogenous. A LGIC ligand that can bind to and activate modified LGICs described herein can be naturally occurring or synthetic. A LGIC ligand that can bind to and activate modified LGICs described herein can be canonical or non-canonical. A LGIC ligand that can bind to and activate modified LGICs described herein can be an agonist or an antagonist. In some aspects, an LGIC ligand is an exogenous LGIC agonist. Examples of LGIC ligands include, without limitation, ACh, nicotine, epibatatine, cytisine, RS56812, tropisetron, nortropisetron, PNU-282987, PHA-543613, compound 0353, compound 0354, compound 0436, compound 0676, compound 702, compound 723, compound 725, granisetron, ivermectin, mequitazine, promazine, varenicline, compound 765, compound 770, 3-(1,4-diazabicyclo[3.2.2]nonan-4-yl)dibenzo[b,d]thiophene 5,5-dioxide, compound 773, and compound 774. In some aspects, an LGIC ligand can be as shown in FIG. 2. In some aspects, an LGIC ligand can be as described elsewhere (see, e.g., WO 2018/009832 and WO 2019/094778).

A LGIC ligand that can bind to and activate modified LGICs described herein can have selective binding (e.g., enhanced binding or increased potency) for a modified LGIC described herein (e.g., relative to an unmodified LGIC). In some aspects, a LGIC ligand that can bind to and activate modified LGICs described herein does not bind to and activate endogenous receptors (e.g., endogenous LGICs). A LGIC ligand that selectively binds to and activates a modified LGIC (e.g., a modified LGIC having at least one amino acid modification that confers pharmacological selectivity to the modified LGIC) described herein over an unmodified LGIC ligand can also be described as having enhanced potency for a modified LGIC. In some aspects, a modified LGIC subunit described herein that selectively binds an exogenous LGIC ligand can have at least 5 fold (e.g., at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 95 fold, at least 100 fold, at least 125 fold, at least 150 fold, at least 200 fold, at least 250 fold, or at least 300 fold) enhanced potency for a modified LGIC. For example, a LGIC ligand that selectively binds to and activates a modified LGIC can have about 10 fold to about 300 fold (e.g., about 10 fold to about 250 fold, about 10 fold to about 200 fold, about 10 fold to about 150 fold, about 10 fold to about 100 fold, about 25 fold to about 300 fold, about 50 fold to about 300 fold, about 100 fold to about 300 fold, about 200 fold to about 300 fold, about 25 fold to about 250 fold, about 50 fold to about 200 fold, or about 100 fold to about 150 fold) enhanced potency for a modified LGIC. In some aspects, a LGIC ligand that binds to and activates a modified LGIC described herein can have a ligand potency of less than 25 nM (e.g., less than 22 nM, less than 20 nM, less than 17 nM, less than 15 nM, less than 13 nM, less than 12 nM, less than 11 nM, less than 10 nM, less than 5 nM, less, than 2 nM, or less than 1 nM). For example, a LGIC ligand that binds to and activates a modified LGIC described herein can have a ligand potency of less than 15 nM. In some aspects, a LGIC ligand can have an EC50 of less than 25 nM (e.g., less than 22 nM, less than 20 nM, less than 17 nM, less than 15 nM, less than 13 nM, less than 12 nM, less than 11 nM, or less than 10 nM) for a modified LGIC subunit described herein.

This document also provides methods of using a modified LGIC described herein and a LGIC ligand that can bind to and activate the modified LGIC as described herein. A LGIC ligand that can bind to and activate the modified LGIC can be used to activate a modified LGIC with temporal and/or spatial control based on delivery of the ligand.

In some aspects, a modified LGIC described herein and a LGIC ligand that can bind to and activate the modified LGIC as described herein can be used to identify a ligand that selectively binds to a modified LGIC described herein. For example, such screening methods can include providing one or more candidate ligands to a modified LGIC described herein, and detecting binding between the candidate ligand and the modified LGIC.

Any appropriate method can be used to detect binding between a candidate ligand and the modified LGIC and any appropriate method can be used to detect activity of a modified LGIC. For example, the ability of a ligand to bind to and activate a modified LGIC can be measured by assays including, but not limited to, membrane potential (MP) assay (e.g., a fluorescence MP assay), radioactive binding assays, and/or voltage clamp measurement of peak currents and sustained currents.

In some aspects, a modified LGIC described herein and a LGIC ligand that can bind to and activate the modified LGIC as described herein can be used to treat a mammal having a channelopathy (e.g., a neural channelopathy or a muscle channelopathy). For example, a mammal having a channelopathy can be treated by administering a modified LGIC described herein, and then administering a LGIC ligand that can bind to and activate the modified LGIC. For example, a mammal having a channelopathy can be treated by administering a modified LGIC described herein (e.g., a modified LGIC including at least one modified LGIC subunit having an α7 nAChR LBD having an amino acid modification at one or more of residues 154, 155, 156, 163, and/or 172, and having a 5HT3 IPD), and then administering a ligand described herein. For example, a mammal having a channelopathy can be treated by administering a modified LGIC described herein including at least one modified LGIC subunit including an α7 nAChR LBD having an amino acid modification at one or more of residues 154, 155, 156, 163, and/or 172 and having a 5HT3 IPD, and then administering varenicline, compound 792, compound 793, and/or compound 817.

Any type of mammal can be treated using a modified LGIC described herein and a LGIC ligand that can bind to and activate the modified LGIC as described herein. For example, humans and other primates such as monkeys can be treated using a modified LGIC described herein and a LGIC ligand that can bind to and activate the modified LGIC as described herein. In some aspects, dogs, cats, horses, cows, pigs, sheep, rabbits, mice, and rats can be treated using a modified LGIC described herein and a LGIC ligand that can bind to and activate the modified LGIC as described herein.

Any appropriate method can be used to identify a mammal having a channelopathy and/or a mammal at risk of developing a channelopathy. For example, genetic testing can be used to identify a mammal having a channelopathy and/or a mammal at risk of developing a channelopathy.

Once identified as having a channelopathy and/or a mammal at risk of developing a channelopathy, the mammal can be administered or instructed to self-administer a modified LGIC described herein (or nucleic acid encoding a modified LGIC subunit described herein such that the encoded subunits can assemble into a modified LGIC described herein), and then administered or instructed to self-administer a LGIC ligand that can bind to and activate the modified LGIC as described herein. A modified LGIC described herein and a LGIC ligand that can bind to and activate the modified LGIC as described herein can be administered together or can be administered separately.

When treating a mammal having a channelopathy and/or a mammal at risk of developing a channelopathy using the materials and methods described herein, the channelopathy can be any channelopathy. As used herein, a channelopathy can be any disease or disorder caused by aberrant ion channel function and/or aberrant ligand function, or which could be alleviated by modulated ion channel function and/or altered cellular ion flux (e.g., calcium ion flux). A channelopathy can be congenital or acquired. Examples of channelopathies include, without limitation, Bartter syndrome, Brugada syndrome, catecholaminergic polymorphic ventricular tachycardia (CPVT), congenital hyperinsulinism, cystic fibrosis, Dravet syndrome, episodic ataxia, erythromelalgia, generalized epilepsy (e.g., with febrile seizures), familial hemiplegic migraine, fibromyalgia, hyperkalemic periodic paralysis, hypokalemic periodic paralysis, Lambert-Eaton myasthenic syndrome, long QT syndrome (e.g., Romano-Ward syndrome), short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, and multiple sclerosis. Alternatively, or in addition, the materials and methods described herein can be used in other applications including, without limitation, pain treatment, cancer cell therapies, appetite control, spasticity treatment, muscle dystonia treatment, tremor treatment, and movement disorder treatment.

In some aspects, a modified LGIC described herein and a LGIC ligand that can bind to and activate the modified LGIC as described herein can be used to modulate the activity of a cell. The activity of the cell that is modulated using a modified LGIC described herein and a LGIC ligand that can bind to and activate the modified LGIC as described herein can be any cellular activity. Examples of cellular activities include, without limitation, active transport (e.g., ion transport), passive transport, excitation, inhibition, ion flux (e.g., calcium ion flux), and exocytosis. The cellular activity can be increased or decreased. For example, a modified LGIC described herein and a LGIC ligand that can bind to and activate the modified LGIC as described herein can be used to modulate (e.g., increase) ion transport across the membrane of a cell. For example, a modified LGIC described herein and a LGIC ligand that can bind to and activate the modified LGIC as described herein can be used to modulate (e.g., increase) the excitability of a cell.

A modified LGIC described herein and a LGIC ligand that can bind to and activate the modified LGIC as described herein can be used to modulate the activity of any type of cell in a mammal. The cell can be a neuron, a glial cell, a myocyte, an immune cell (e.g., neutrophils, eosinophils, basophils, lymphocytes, and monocytes), an endocrine cell, or a stem cell (e.g., an embryonic stem cell). In some aspects, the cell can be an excitable cell. The cell can be in vivo or ex vivo.

A modified LGIC described herein (or nucleic acid encoding a modified LGIC subunit described herein such that the encoded subunits can assemble into a modified LGIC described herein) can be administered by any appropriate method. A modified LGIC can be administered as modified LGIC subunits or as pre-assembled modified LGICs. A modified LGIC can be administered as a nucleic acid encoding a modified LGIC. A modified LGIC can be administered as a nucleic acid encoding a modified LGIC subunit described herein. For example, a nucleic acid can be delivered as naked nucleic acid or using any appropriate vector (e.g., a recombinant vector). Vectors can be a DNA based vector, an RNA based, or combination thereof. Vectors can express a nucleic acid in dividing cells or non-dividing cells. Examples of recombinant vectors include, without limitation, plasmids, viral vectors (e.g., retroviral vectors, adenoviral vectors, adeno-associated viral vectors, and herpes simplex vectors), cosmids, and artificial chromosomes (e.g., yeast artificial chromosomes or bacterial artificial chromosomes). In some aspects, a nucleic acid encoding a modified LGIC subunit described herein can be expressed by an adeno-associated viral vector.

A modified LGIC described herein can be detected (e.g., to confirm its presence in a cell) by any appropriate method. In some aspects, an agent that selectively binds a modified LGIC can be used to detect the modified LGIC. Examples of agents that can be used to bind to a modified LGIC described herein include, without limitation, antibodies, proteins (e.g., bungarotoxin), and small molecule ligands (e.g., PET ligands). An agent that selectively binds a modified LGIC can include a detectable label (e.g., fluorescent labels, radioactive labels, positron emitting labels, and enzymatic labels). Methods to detect LGIC expression in a cell can include fluorescence imaging, autoradiography, functional MRI, PET, and SPECT.

A modified LGIC described herein and a LGIC ligand that can bind to and activate the modified LGIC as described herein can be administered to a mammal having a channelopathy and/or at risk of developing a channelopathy as a combination therapy with one or more additional agents/therapies used to treat a channelopathy. For example, a combination therapy used to treat a mammal having a channelopathy as described herein can include administering a modified LGIC described herein and a LGIC ligand that can bind to and activate the modified LGIC as described herein and treating with acetazolaminde, dichlorphenamide, mexilitine, glucose, calcium gluconate, L-DOPA, muscle stimulation, spinal stimulation, brain stimulation, and/or nerve stimulation.

In embodiments where a modified LGIC described herein and a LGIC ligand that can bind to and activate the modified LGIC as described herein are used in combination with additional agents/therapies used to treat a channelopathy, the one or more additional agents can be administered at the same time or independently. For example, a modified LGIC described herein and a LGIC ligand that can bind to and activate the modified LGIC as described herein first, and the one or more additional agents administered second, or vice versa. In embodiments where a modified LGIC described herein and a LGIC ligand that can bind to and activate the modified LGIC as described herein are used in combination with one or more additional therapies used to treat a channelopathy, the one or more additional therapies can be performed at the same time or independently of the administration of a modified LGIC described herein and a LGIC ligand that can bind to and activate the modified LGIC as described herein. For example, a modified LGIC described herein and a LGIC ligand that can bind to and activate the modified LGIC as described herein can be administered before, during, or after the one or more additional therapies are performed.

In some aspects, a modified LGIC described herein and/or a LGIC ligand that can bind to and activate the modified LGIC as described herein can be formulated into a pharmaceutically acceptable composition for administration to a mammal having a channelopathy or at risk of developing a channelopathy. For example, a therapeutically effective amount of a modified LGIC described herein (e.g., a nucleic acid encoding a modified LGIC described herein) and/or a LGIC ligand that can bind to and activate the modified LGIC as described herein can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing a modified LGIC described herein and/or a LGIC ligand that can bind to and activate the modified LGIC as described herein can be designed for oral, parenteral (including subcutaneous, intracranial, intraarterial, intramuscular, intravenous, intracoronary, intradermal, or topical), or inhaled administration. When being administered orally, a pharmaceutical composition containing a therapeutically effective amount of a modified LGIC described herein (e.g., a nucleic acid encoding a modified LGIC described herein) and/or a LGIC ligand that can bind to and activate the modified LGIC as described herein can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. Compositions for inhalation can be delivered using, for example, an inhaler, a nebulizer, and/or a dry powder inhaler. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A pharmaceutically acceptable composition including a therapeutically effective amount of a modified LGIC described herein (e.g., a nucleic acid encoding a modified LGIC described herein) and/or a LGIC ligand that can bind to and activate the modified LGIC as described herein can be administered locally or systemically. In some aspects, a composition containing a therapeutically effective amount of a modified LGIC described herein (e.g., a nucleic acid encoding a modified LGIC described herein) and/or a LGIC ligand that can bind to and activate the modified LGIC as described herein can be administered systemically by venous or oral administration to, or inhalation by a mammal (e.g., a human). In some aspects, a composition containing a therapeutically effective amount of a modified LGIC described herein (e.g., a nucleic acid encoding a modified LGIC described herein) and/or a LGIC ligand that can bind to and activate the modified LGIC as described herein can be administered locally by percutaneous, subcutaneous, intramuscular, intracranial, or open surgical administration (e.g., injection) to a target tissue of a mammal (e.g., a human).

Effective doses can vary depending on the severity of the channelopathy, the route of administration, the age and general health condition of the subject, excipient usage, the possibility of co-usage with other therapeutic treatments such as use of other agents, and the judgment of the treating physician.

The frequency of administration can be any frequency that improves symptoms of a channelopathy without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing a therapeutically effective amount of a modified LGIC described herein (e.g., a nucleic acid encoding a modified LGIC described herein) and/or a LGIC ligand that can bind to and activate the modified LGIC as described herein can include rest periods. For example, a composition containing a therapeutically effective amount of a modified LGIC described herein (e.g., a nucleic acid encoding a modified LGIC described herein) and/or a LGIC ligand that can bind to and activate the modified LGIC as described herein can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the channelopathy may require an increase or decrease in administration frequency.

An effective duration for administering a composition containing a therapeutically effective amount of a modified LGIC described herein (e.g., a nucleic acid encoding a modified LGIC described herein) and/or a LGIC ligand that can bind to and activate the modified LGIC as described herein can be any duration that improves symptoms of a channelopathy without producing significant toxicity to the mammal. For example, the effective duration can vary from several days to several weeks, months, or years. In some aspects, the effective duration for the treatment of a channelopathy can range in duration from about one month to about 10 years. Multiple factors can influence the actual effective duration used for a particular treatment. For example, an effective duration can vary with the frequency of administration, effective amount, use of multiple treatment agents, route of administration, and severity of the channelopathy being treated.

In certain instances, a course of treatment and the symptoms of the mammal being treated for a channelopathy can be monitored. Any appropriate method can be used to monitor the symptoms of a channelopathy.

The invention will be further described in the following examples, which do not limit the scope of the invention described in the claims.

### EXAMPLES

### Example 1: Potency-Enhancing Ligand Binding Domain Mutations in a PSAM4-5HT3 Chimeric LGIC Subunit

PSAM4-5HT3 (α7-5HT3^{L131G,Q139L,Y217F}) LGICs having additional mutations were evaluated.

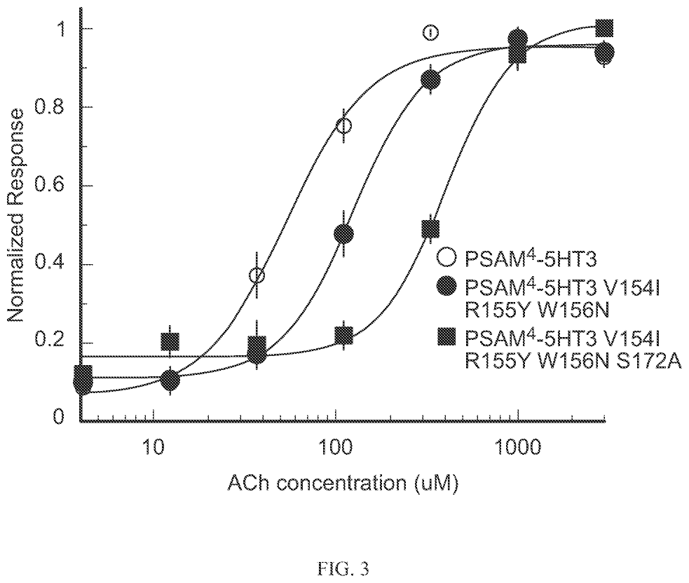

Dose response in PSAM4-5HT3 (α7-5HT3^{L131G,Q139L,Y217F}) LGICs having additional mutations was analyzed. Results showed right-shifted ACh potency with additional mutations to PSAM4-5HT3. Additional mutations at residues 154, 155, and 156 right-shift ACh potency (FIG. 3). Adding another mutation at residue 172 further right-shifted the ACh dose response (FIG. 3).

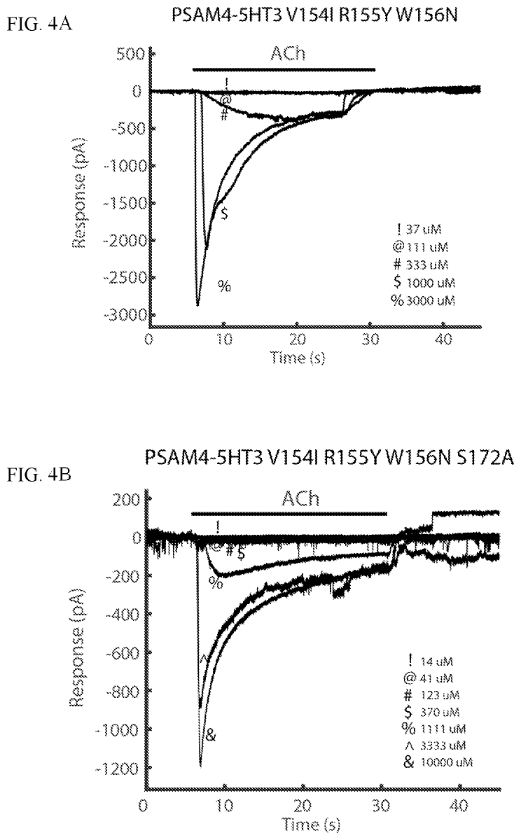

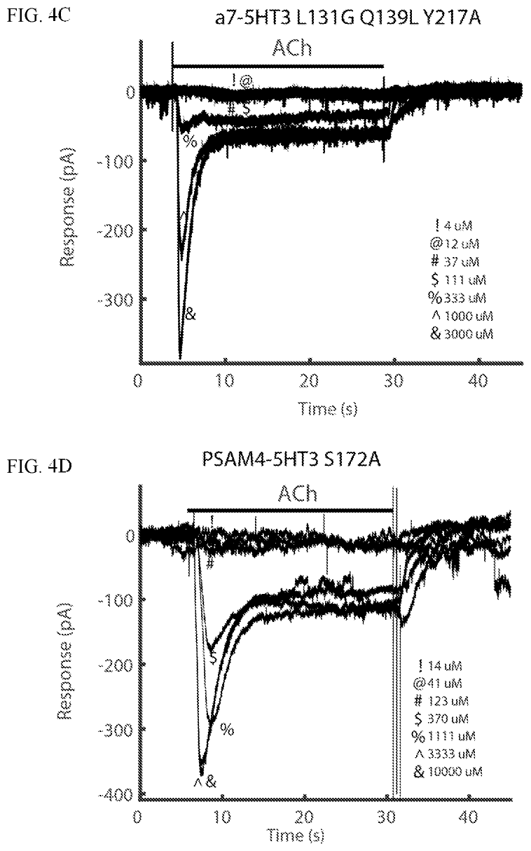

Electrophysiological response in PSAM⁴-5HT3 (α7-5HT3^{L131G,Q139L,Y217F}) LGICs having additional mutations was analyzed. Electrophysiological recordings in HEK 293 cells expressing chimeric ion channels with mutations to PSAM4-5HT3 showed current responses to ACh concentrations. Electrophysiological measurements are shown for PSAM4-5HT3 with mutations at additional residues 154, 155, 156, and/or 172 (FIGS. 4A, 4B, and 4D). Dose response for ACh is also shown for PSAM4-5HT3 (FIG. 4C).

TABLE 2
EC50s (SEM) for additional mutations of α7^{L131G, Q139L, Y217F} -5HT3 (μM).

| | Acetylcholine | | Choline | | Varenicline | | uPSEM 792 | | uPSEM 793 | | uPSEM 817 | |
|---|---|---|---|---|---|---|---|---|---|---|---|---|
| PSAM4-5HT3 | 64 | (8) | 143 | (21) | 0.0036 | (0.0001) | 0.0039 | (0.000060 | 0.0017 | (0.0005) | 0.0041 | (0.001) |
| V154I, R155Y, W156N | 129 | (15) | 330 | (25) | 0.0085 | (0.008) | 0.0085 | (0.0015) | 0.0012 | (0.0005) | 0.013 | (0.0009) |
| V154L, R155Y, W156Q | 49 | (1) | 94 | (11) | 0.0075 | (0.0025) | 0.0126 | (0.0002) | 0.0007 | (0.00001) | 0.017 | (0.0005) |
| W156N | 84 | (7) | 172 | (49) | 0.0075 | (0.0038) | 0.011 | (0.0003) | 0.0007 | (0.00001) | 0.015 | (0.003) |
| W156N, H163T | 189 | (45) | 646 | (127) | 0.009 | (0.002) | 0.013 | (0.004) | 0.002 | (0.001) | 0.013 | (0.002) |
| H163A | 96 | (7) | 219 | (39) | 0.0054 | (0.0006) | 0.0051 | (0.0046) | 0.0005 | (0.0001) | 0.053 | (0.044) |
| H163D | 104 | (8) | 361 | (40) | 0.0038 | | 0.012 | | 0.0003 | | 0.0025 | |
| H163F | 98 | (19) | 230 | (43) | 0.0061 | (0.0016) | 0.0098 | (0.0038) | 0.0007 | (0.0002) | 0.0082 | (0.0007) |
| H163K | 87 | (12) | 168 | (27) | 0.0050 | (0.0001) | 0.0086 | (0.0013) | 0.0005 | (0.0001) | 0.01 | (0.006) |
| H163N | 73 | (25) | 107 | (18) | 0.0036 | (0.0007) | 0.0063 | (0.0006) | 0.0021 | (0.0017) | 0.0091 | (0.0013) |
| H163R | 82 | (11) | 153 | (29) | 0.0061 | (0.0008) | 0.0072 | (0.001) | 0.0015 | (0.001) | 0.0055 | (0.0015) |
| H163S | 99 | (5) | 245 | (2) | 0.0064 | (0.0011) | 0.0086 | (0.0014) | 0.0004 | (0.0001) | 0.0081 | (0.0022) |
| H163T | 123 | (5) | 330 | (10) | 0.024 | (0.019) | 0.012 | (0.007) | 0.0014 | (0.0008) | 0.012 | (0.001) |
| H163V | 179 | (33) | 508 | (77) | 0.0073 | (0.0037) | 0.0072 | (0.0011) | 0.0015 | (0.001) | 0.009 | (0.002) |
| S172A | 175 | (23) | 349 | (28) | 0.0049 | (0.0006) | 0.0064 | (0.0016) | 0.004 | (0.0004) | 0.007 | (0.001) |
| H163T, S172A | 435 | (19) | 1016 | (220) | 0.0057 | (0.0024) | 0.011 | (0.004) | 0.0025 | (0.0011) | 0.0078 | (0.0020) |
| V154I, R155Y, W156N, S172A | 367 | (3) | 680 | (55) | 0.011 | (0.005) | 0.015 | (0.005) | 0.0041 | (0.0014) | 0.012 | (0.003) |
| V154I, R155Y, W156N, S172V | 279 | (66) | 910 | | 0.013 | (0.003) | 0.025 | (0.009) | 0.0077 | (0.035) | 0.013 | (0.002) |
| H163T, S172V | 496 | (112) | 1330 | | 0.009 | (0.0001) | 0.0054 | (0.0039) | 0.0015 | (0.0005) | 0.0063 | (0.002) |
| S172I | 297 | (63) | 1114 | (114) | 0.015 | (0.010) | 0.012 | (0.003) | 0.010 | (0.001) | 0.018 | (0.003) |
| S172V | 196 | (20) | 895 | (42) | 0.0038 | (0.0014) | 0.0043 | (0.0012) | 0.0027 | (0.0001) | 0.0060 | (0.0007) |
| L131G, Q139L, Y217F | 64 | (8) | 143 | (21) | 0.0036 | (0.0001) | 0.0039 | (0.000060 | 0.0017 | (0.0005) | 0.0041 | (0.001) |

| L131G, Q139L, W171F | 510 | | 177 | | >0.1 | >0.1 | | >0.1 | | >0.1 | |
|---|---|---|---|---|---|---|---|---|---|---|---|
| L131G, Q139L, W171Y | 430 | | — | | >0.1 | >0.1 | | >0.1 | | >0.1 | |
| L131G, Q139L, Y217A | 392 | (62) | 832 | (329) | >0.1 | 0.022 | (0.001) | 0.0012 | (0.0001) | 0.0092 | (0.0048) |
| L131G, Q139L, Y217H | 1300 | (100) | 2065 | (935) | >0.1 | 0.0122 | (0.0002) | 0.0008 | (0.0001) | 0.016 | (0.003) |

| L131G, Q139L, Y217N | 213 | | — | | >0.1 | >0.1 | >0.1 | | >0.1 | |
|---|---|---|---|---|---|---|---|---|---|---|
| L131G, Q139L, Y217V | 246 | (33) | 1165 | (65) | >0.1 | >0.1 | 0.0024 | (0.0001) | 0.024 | (0.001) |

Example 2: Exemplary Sequences

SEQ ID NO: 1
human α7 nAChR LBD
MRCSPGGVWLALAASLLHVSLQGEFQRKLYKELVKNYNPLERPVANDSQPLTVYFSLSLLQI
MDVDEKNQVLTTNIWLQMSWTDHYLQWNVSEYPGVKTVRFPDGQIWKPDILLYNSADERFDA
TFHTNVLVNSSGHCQYLPPGIFKSSCYIDVRWFPFDVQHCKLKFGSWSYGGWSLDLQMQEAD
ISGYIPNGEWDLVGIPGKRSERFYECCKEPYPDVTFTV
SEQ ID NO: 2
human α7 nAChR LBD
MRCSPGGVWLALAASLLHVSLQGEFQRKLYKELVKNYNPLERPVANDSQPLTVYFSLSLLQI
MDVDEKNQVLTTNIWLQMSWTDHYLQWNVSEYPGVKTVRFPDGQIWKPDILLYNSADERFDA
TFHTNVLVNSSGHCQYLPPGIFKSSCYIDVRWFPFDVQHCKLKFGSWSYGGWSLDLQMQEAD
ISGYIPNGEWDLVGIPGKRSERFYECCKEPYPDVTFTVTMRRR
SEQ ID NO: 3
human α7 nAChR LBD
MRCSPGGVWLALAASLLHVSLQGEFQRKLYKELVKNYNPLERPVANDSQPLTVYFSLSLLQI
MDVDEKNQVLTTNIWLQMSWTDHYLQWNVSEYPGVKTVRFPDGQIWKPDILLYNSADERFDA
TFHTNVLVNSSGHCQYLPPGIFKSSCYIDVRWFPFDVQHCKLKFGSWSYGGWSLDLQMQEAD
ISGYIPNGEWDLVGIPGKRSERFYECCKEPYPDVTFTVTMRRRTLYY
SEQ ID NO: 4
rat α7 nAChR amino acid sequence (including both a LBD and an IPD)
MGGGRGGIWLALAAALLHVSLQGEFQRRLYKELVKNYNPLERPVANDSQPLTVYFSLSLLQI
MDVDEKNQVLTTNIWLQMSWTDHYLQWNMSEYPGVKNVRFPDGQIWKPDILLYNSADERFDA
TFHTNVLVNASGHCQYLPPGIFKSSCYIDVRWFPFDVQQCKLKFGSWSYGGWSLDLQMQEAD
ISSYIPNGEWDLMGIPGKRNEKFYECCKEPYPDVTYTVTMRRRTLYYGLNLLIPCVLISALA
LLVFLLPADSGEKISLGITVLLSLTVEMLLVAEIMPATSDSVPLIAQYFASTMIIVGLSVVV
TVIVLRYHHHDPDGGKMPKWTRIILLNWCAWFLRMKRPGEDKVRPACQHKPRRCSLASVELS
AGAGPPTSNGNLLYIGFRGLEGMHCAPTPDSGVVCGRLACSPTHDEHLMHGAHPSDGDPDLA
KILEEVRYIANRNRCQDESEVICSEWKFAACVVDPLCLMAFSVFTIICTIGILMSAPNFVEA
VSKDFA
SEQ ID NO: 5
murine 5HT3 IPD
IIRRRPLFYAVSLLLPSIFLMVVDIVGFCLPPDSGERVSFKITLLLGYSVFLIIVSDTLPAT
IGTPLIGVYFVVCMALLVISLAETIFIVRLVHKQDLQRPVPDWLRHLVLDRIAWILCLGEQP
MAHRPPATFQANKTDDCSGSDLLPAMGNHCSHVGGPQDLEKTPRGRGSPLPPPREASLAVRG
LLQELSSIRHELEKRDEMREVARDWLRVGYVLDRLLFRIYLLAVLAYSITLVTLWSIWHYS
SEQ ID NO: 6
human 5HT3 IPD
IIRRRPLFYVVSLLLPSIFLMVMDIVGFYLPPNSGERVSFKITLLLGYSVFLIIVSDTLPAT
AIGTPLIGVYFVVCMALLVISLAETIFIVRLVHKQDLQQPVPAWLRHLVLERIAWLLCLREQ
STSQRPPATSQATKTDDCSAMGNHCSHMGGPQDFEKSPRDRCSPPPPPREASLAVCGLLQEL
SSIRQFLEKRDEIREVARDWLRVGSVLDKLLFHIYLLAVLAYSITLVMLWSIWQYA
SEQ ID NO: 7
amino acid sequence of α7-5HT3 chimeric receptor including a human α7
nAChR LBD and a murine 5HT3 IPD
MRCSPGGVWLALAASLLHVSLQGEFQRKLYKELVKNYNPLERPVANDSQPLTVYFSLSLLQI
MDVDEKNQVLTTNIWLQMSWTDHYLQWNVSEYPGVKTVRFPDGQIWKPDILLYNSADERFDA
TFHTNVLVNSSGHCQYLPPGIFKSSCYIDVRWFPFDVQHCKLKFGSWSYGGWSLDLQMQEAD
ISGYIPNGEWDLVGIPGKRSERFYECCKEPYPDVTFTVIIRRRPLFYAVSLLLPSIFLMVVD
IVGFCLPPDSGERVSFKITLLLGYSVFLIIVSDTLPATIGTPLIGVYFVVCMALLVISLAET
IFIVRLVHKQDLQRPVPDWLRHLVLDRIAWILCLGEQPMAHRPPATFQANKTDDCSGSDLLP
AMGNHCSHVGGPQDLEKTPRGRGSPLPPPREASLAVRGLLQELSSIRHFLEKRDEMREVARD
WLRVGYVLDRLLFRIYLLAVLAYSITLVTLWSIWHYS
SEQ ID NO: 8
amino acid sequence of α7-5HT3 chimeric receptor including human α7
nAChR LBD and a human 5HT3 IPD
MRCSPGGVWLALAASLLHVSLQGEFQRKLYKELVKNYNPLERPVANDSQPLTVYFSLSLLQI
MDVDEKNQVLTTNIWLQMSWTDHYLQWNVSEYPGVKTVRFPDGQIWKPDILLYNSADERFDA
TFHTNVLVNSSGHCQYLPPGIFKSSCYIDVRWFPFDVQHCKLKFGSWSYGGWSLDLQMQEAD
ISGYIPNGEWDLVGIPGKRSERFYECCKEPYPDVTFTVIIRRRPLFYVVSLLLPSIFLMVMD
IVGFYLPPNSGERVSFKITLLLGYSVFLIIVSDTLPATAIGTPLIGVYFVVCMALLVISLAE
TIFIVRLVHKQDLQQPVPAWLRHLVLERIAWLLCLREQSTSQRPPATSQATKTDDCSAMGNH
CSHMGGPQDFEKSPRDRCSPPPPPREASLAVCGLLQELSSIRQFLEKRDEIREVARDWLRVG
SVLDKLLFHIYLLAVLAYSITLVMLWSIWQYA
SEQ ID NO: 9
ER export sequence
FCYENEV
SEQ ID NO: 10
CHRNB4 signal sequence
MRRAPSLVLFFLVALCGRGNC
SEQ ID NO: 11
KCNB1 somatic targeting sequence
QSQPILNTKEMAPQSKPPEELEMSSMPSPVAPLPARTEGVIDMRSMSSIDSFISCATDEPEA
TRF
SEQ ID NO: 12
nucleic acid sequence encoding a rat α7 nAChR amino acid sequence
(including both a LBD and an IPD)
ATGGGCGGCGGGCGGGGAGGCATCTGGCTGGCTCTGGCCGCGGCGCTGCTGCACGTGTCCCT
GCAAGGCGAGTTCCAGAGGAGGCTGTACAAGGAGCTGGTCAAGAACTACAACCCGCTGGAGA
GGCCGGTGGCCAACGACTCGCAGCCGCTCACCGTGTACTTCTCCCTGAGTCTCCTGCAGATC
ATGGATGTGGATGAGAAGAACCAAGTTTTAACCACCAACATTTGGCTACAAATGTCTTGGAC
AGATCACTATTTGCAGTGGAACATGTCTGAGTACCCCGGAGTGAAGAATGTTCGTTTTCCAG
ATGGCCAGATTTGGAAACCAGACATTCTCCTCTATAACAGTGCTGATGAGCGCTTTGATGCC
ACGTTCCACACCAATGTTTTGGTGAATGCATCTGGGCATTGCCAGTATCTCCCTCCAGGCAT
ATTCAAGAGCTCCTGCTACATTGACGTTCGCTGGTTCCCTTTTGATGTGCAGCAGTGCAAAC
TGAAGTTTGGGTCCTGGTCCTATGGAGGGTGGTCACTGGACCTGCAAATGCAAGAGGCAGAT
ATCAGCAGCTATATCCCCAACGGAGAATGGGATCTCATGGGAATCCCTGGCAAAAGGAATGA
GAAGTTCTATGAGTGCTGCAAAGAGCCATACCCAGATGTCACCTACACAGTAACCATGCGCC
GTAGGACACTCTACTATGGCCTCAATCTGCTCATCCCTTGTGTACTCATTTCAGCCCTGGCT
CTGCTGGTATTCTTGCTGCCTGCAGACTCTGGAGAGAAAATCTCTCTTGGAATAACTGTCTT
ACTTTCTCTGACTGTCTTCATGCTGCTTGTGGCTGAGATCATGCCAGCAACATCTGATTCTG
TGCCCTTGATAGCACAATACTTCGCCAGCACCATGATCATCGTGGGCCTCTCTGTAGTGGTG
ACAGTGATTGTGCTGAGATATCACCACCATGACCCTGATGGTGGCAAAATGCCTAAGTGGAC
CAGAATCATTCTCCTGAACTGGTGTGCATGGTTTCTGCGCATGAAGAGGCCCGGAGAGGACA
AGGTGCGGCCAGCTTGTCAGCACAAGCCTCGGCGCTGCAGCCTGGCCAGTGTGGAGCTGAGT
GCAGGTGCTGGGCCACCCACCAGCAATGGCAACCTGCTCTACATTGGCTTCCGAGGCCTGGA
GGGCATGCACTGTGCCCCAACTCCAGACTCTGGGGTCGTATGTGGCCGTTTGGCCTGCTCCC
CAACACATGATGAGCACCTCATGCACGGTGCACACCCCTCTGATGGGGACCCCGACCTGGCC
AAGATCCTGGAGGAGGTCCGCTACATCGCCAACCGCAACCGCTGCCAGGACGAGAGTGAGGT
GATCTGCAGTGAATGGAAGTTTGCAGCCTGCGTGGTGGACCCGCTTTGCCTCATGGCCTTTT
CGGTCTTTACCATCATCTGTACCATCGGCATCCTCATGTCAGCTCCAAACTTTGTGGAGGCT
GTGTCCAAAGACTTTGCTTAA
SEQ ID NO: 13
nucleic acid sequence encoding a human α7 nAChR LBD
ATGCGCTGTTCTCCAGGCGGCGTGTGGCTCGCCCTGGCTGCTTCCCTTCTGCACGTTAGCCT
GCAGGGTGAGTTCCAGCGCAAACTGTATAAGGAGCTTGTTAAGAATTATAACCCCCTGGAGC
GGCCGGTCGCAAATGATTCCCAGCCACTGACAGTGTACTTCAGCCTCTCCTTGCTGCAGATC
ATGGACGTGGATGAAAAGAACCAGGTGCTGACCACTAATATTTGGTTGCAGATGTCCTGGAC
CGATCACTACTTGCAGTGGAATGTGAGCGAATACCCAGGTGTAAAGACTGTAAGATTCCCTG
ACGGCCAAATCTGGAAACCAGATATCCTGCTGTACAACAGCGCAGACGAAAGGTTTGATGCA
ACATTTCACACCAACGTGTTGGTCAATTCTTCAGGCCACTGCCAGTACCTGCCCCCTGGAAT
CTTCAAGTCCTCATGCTATATCGACGTCCGCTGGTTTCCCTTCGACGTCCAGCACTGCAAAC
TCAAATTCGGGAGCTGGAGCTACGGCGGATGGAGCCTGGATCTGCAAATGCAGGAGGCTGAC
ATCTCTGGTTACATCCCGAATGGGGAGTGGGACCTTGTGGGAATCCCCGGTAAAAGAAGCGA
GCGATTTTATGAATGCTGCAAGGAACCCTACCCTGACGTAACATTCACAGTT
SEQ ID NO: 14
nucleic acid sequence encoding a human α7 nAChR LBD
ATGCGCTGTTCTCCAGGCGGCGTGTGGCTCGCCCTGGCTGCTTCCCTTCTGCACGTTAGCCT
GCAGGGTGAGTTCCAGCGCAAACTGTATAAGGAGCTTGTTAAGAATTATAACCCCCTGGAGC
GGCCGGTCGCAAATGATTCCCAGCCACTGACAGTGTACTTCAGCCTCTCCTTGCTGCAGATC
ATGGACGTGGATGAAAAGAACCAGGTGCTGACCACTAATATTTGGTTGCAGATGTCCTGGAC
CGATCACTACTTGCAGTGGAATGTGAGCGAATACCCAGGTGTAAAGACTGTAAGATTCCCTG
ACGGCCAAATCTGGAAACCAGATATCCTGCTGTACAACAGCGCAGACGAAAGGTTTGATGCA
ACATTTCACACCAACGTGTTGGTCAATTCTTCAGGCCACTGCCAGTACCTGCCCCCTGGAAT
CTTCAAGTCCTCATGCTATATCGACGTCCGCTGGTTTCCCTTCGACGTCCAGCACTGCAAAC
TCAAATTCGGGAGCTGGAGCTACGGCGGATGGAGCCTGGATCTGCAAATGCAGGAGGCTGAC
ATCTCTGGTTACATCCCGAATGGGGAGTGGGACCTTGTGGGAATCCCCGGTAAAAGAAGCGA
GCGATTTTATGAATGCTGCAAAGAGCCCTACCCAGATGTCACCTTCACAGTGACCATGCGGA
GACGC
SEQ ID NO: 15
nucleic acid sequence encoding a human α7 nAChR LBD
ATGCGCTGTTCTCCAGGCGGCGTGTGGCTCGCCCTGGCTGCTTCCCTTCTGCACGTTAGCCT
GCAGGGTGAGTTCCAGCGCAAACTGTATAAGGAGCTTGTTAAGAATTATAACCCCCTGGAGC
GGCCGGTCGCAAATGATTCCCAGCCACTGACAGTGTACTTCAGCCTCTCCTTGCTGCAGATC
ATGGACGTGGATGAAAAGAACCAGGTGCTGACCACTAATATTTGGTTGCAGATGTCCTGGAC
CGATCACTACTTGCAGTGGAATGTGAGCGAATACCCAGGTGTAAAGACTGTAAGATTCCCTG
ACGGCCAAATCTGGAAACCAGATATCCTGCTGTACAACAGCGCAGACGAAAGGTTTGATGCA
ACATTTCACACCAACGTGTTGGTCAATTCTTCAGGCCACTGCCAGTACCTGCCCCCTGGAAT
CTTCAAGTCCTCATGCTATATCGACGTCCGCTGGTTTCCCTTCGACGTCCAGCACTGCAAAC
TCAAATTCGGGAGCTGGAGCTACGGCGGATGGAGCCTGGATCTGCAAATGCAGGAGGCTGAC
ATCTCTGGTTACATCCCGAATGGGGAGTGGGACCTTGTGGGAATCCCCGGTAAAAGAAGCGA
GCGATTTTATGAATGCTGCAAAGAGCCCTACCCAGATGTCACCTTCACAGTGACCATGCGGA
GACGCACACTGTATTAC
SEQ ID NO: 16
nucleic acid sequence encoding a murine 5HT3 IPD
ATCATCAGAAGAAGGCCATTGTTCTACGCCGTTAGTTTGTTGCTCCCCAGTATTTTTCTCAT
GGTCGTGGACATCGTGGGATTTTGTCTCCCACCTGATAGCGGGGAGAGGGTCTCCTTTAAGA
TTACCTTGTTGCTCGGCTATTCTGTATTTCTGATCATCGTGTCCGATACCCTTCCTGCCACA
ATCGGCACTCCGCTGATAGGAGTGTATTTCGTCGTGTGTATGGCACTCCTGGTGATAAGTCT
GGCGGAAACTATCTTCATTGTACGGCTGGTACATAAGCAGGACCTGCAAAGACCCGTGCCAG
ACTGGTTGCGACACCTTGTGCTGGACAGAATTGCATGGATTCTGTGTCTTGGCGAGCAACCT
ATGGCCCACCGGCCACCTGCAACCTTTCAAGCCAACAAGACAGACGATTGTAGTGGGTCTGA
TCTGTTGCCTGCTATGGGGAATCACTGCTCCCATGTTGGGGGACCACAAGATTTGGAAAAGA
CCCCACGGGGGCGGGGATCACCCCTTCCTCCTCCCCGAGAAGCCTCTCTCGCTGTCCGGGGG
CTGCTCCAGGAACTGTCAAGCATCCGACATTTTCTGGAGAAGCGGGACGAGATGAGGGAAGT
CGCTAGAGACTGGCTGCGAGTGGGCTACGTCCTTGACAGGCTGCTGTTTCGGATCTACTTGC
TGGCGGTGCTGGCTTATTCCATTACTCTGGTGACACTCTGGTCCATATGGCACTACAGTTAG
SEQ ID NO: 17
nucleic acid sequence encoding a human 5HT3 IPD
ATCATCCGTAGAAGGCCTCTGTTTTACGTGGTGAGCCTGCTGCTGCCATCCATCTTCCTGAT
GGTCATGGACATCGTGGGCTTTTACCTGCCACCCAATTCTGGCGAGCGCGTGAGCTTCAAGA
TCACACTGCTGCTGGGCTATAGCGTGTTTCTGATCATCGTGTCCGATACCCTGCCTGCAACA
GCAATCGGAACCCCACTGATCGGCGTGTATTTCGTGGTGTGCATGGCCCTGCTGGTCATCAG
CCTGGCCGAGACAATCTTTATCGTGCGGCTGGTGCACAAGCAGGACCTGCAGCAGCCTGTGC
CAGCATGGCTGAGGCACCTGGTGCTGGAGAGGATCGCATGGCTGCTGTGCCTGAGAGAGCAG
TCCACATCTCAGAGGCCTCCAGCCACCTCTCAGGCCACCAAGACAGACGATTGCTCTGCCAT
GGGCAATCACTGTAGCCACATGGGCGGCCCCCAGGACTTTGAGAAGTCCCCTCGCGATCGGT
GCTCTCCACCTCCACCACCTAGGGAGGCCAGCCTGGCCGTGTGCGGCCTGCTGCAGGAGCTG
TCCTCTATCCGGCAGTTCCTGGAGAAGCGCGACGAGATCCGGGAGGTGGCCAGAGATTGGCT
GAGGGTGGGCAGCGTGCTGGATAAGCTGCTGTTTCACATCTACCTGCTGGCAGTCCTGGCCT
ATTCTATTACCCTGGTCATGCTGTGGTCCATCTGGCAGTACGCC
SEQ ID NO: 18
nucleic acid sequence encoding a α7-5HT3 chimeric receptor including a
human α7 nAChR LBD and a murine 5HT3 IPD
ATGCGCTGTTCTCCAGGCGGCGTGTGGCTCGCCCTGGCTGCTTCCCTTCTGCACGTTAGCCT
GCAGGGTGAGTTCCAGCGCAAACTGTATAAGGAGCTTGTTAAGAATTATAACCCCCTGGAGC
GGCCGGTCGCAAATGATTCCCAGCCACTGACAGTGTACTTCAGCCTCTCCTTGCTGCAGATC
ATGGACGTGGATGAAAAGAACCAGGTGCTGACCACTAATATTTGGTTGCAGATGTCCTGGAC
CGATCACTACTTGCAGTGGAATGTGAGCGAATACCCAGGTGTAAAGACTGTAAGATTCCCTG
ACGGCCAAATCTGGAAACCAGATATCCTGCTGTACAACAGCGCAGACGAAAGGTTTGATGCA
ACATTTCACACCAACGTGTTGGTCAATTCTTCAGGCCACTGCCAGTACCTGCCCCCTGGAAT
CTTCAAGTCCTCATGCTATATCGACGTCCGCTGGTTTCCCTTCGACGTCCAGCACTGCAAAC
TCAAATTCGGGAGCTGGAGCTACGGCGGATGGAGCCTGGATCTGCAAATGCAGGAGGCTGAC
ATCTCTGGTTACATCCCGAATGGGGAGTGGGACCTTGTGGGAATCCCCGGTAAAAGAAGCGA
GCGATTTTATGAATGCTGCAAGGAACCCTACCCTGACGTAACATTCACAGTTATCATCAGAA
GAAGGCCATTGTTCTACGCCGTTAGTTTGTTGCTCCCCAGTATTTTTCTCATGGTCGTGGAC
ATCGTGGGATTTTGTCTCCCACCTGATAGCGGGGAGAGGGTCTCCTTTAAGATTACCTTGTT
GCTCGGCTATTCTGTATTTCTGATCATCGTGTCCGATACCCTTCCTGCCACAATCGGCACTC
CGCTGATAGGAGTGTATTTCGTCGTGTGTATGGCACTCCTGGTGATAAGTCTGGCGGAAACT
ATCTTCATTGTACGGCTGGTACATAAGCAGGACCTGCAAAGACCCGTGCCAGACTGGTTGCG
ACACCTTGTGCTGGACAGAATTGCATGGATTCTGTGTCTTGGCGAGCAACCTATGGCCCACC
GGCCACCTGCAACCTTTCAAGCCAACAAGACAGACGATTGTAGTGGGTCTGATCTGTTGCCT
GCTATGGGGAATCACTGCTCCCATGTTGGGGGACCACAAGATTTGGAAAAGACCCCACGGGG
GCGGGGATCACCCCTTCCTCCTCCCCGAGAAGCCTCTCTCGCTGTCCGGGGGCTGCTCCAGG
AACTGTCAAGCATCCGACATTTTCTGGAGAAGCGGGACGAGATGAGGGAAGTCGCTAGAGAC
TGGCTGCGAGTGGGCTACGTCCTTGACAGGCTGCTGTTTCGGATCTACTTGCTGGCGGTGCT
GGCTTATTCCATTACTCTGGTGACACTCTGGTCCATATGGCACTACAGTTAG
SEQ ID NO: 19
nucleic acid sequence encoding an ER export sequence
TTTTGCTATGAAAACGAAGTC
SEQ ID NO: 20
nucleic acid sequence encoding a CHRNB4 signal sequence
ATGAGAAGGGCCCCATCCCTGGTATTGTTTTTTTTGGTAGCTTTGTGCGGGAGGGGGAACTG
C
SEQ ID NO: 21
nucleic acid sequence encoding a KCNB1 somatic targeting sequence
CAAAGCCAACCTATCCTTAACACTAAAGAGAGCGCCGCTCAATCCAAACCCAAAGAAGAGTT
GGAAATGGAGTCTATACCTTCACCTGTTGCACCTCTCCCTACTAGGACCGAAGGCGTGATTG
ACATGCGCTCTATGTCTAGTATAGATAGCTTTATATCCTGCGCCACAGACTTTCCCGAAGCC
ACTAGGTTC

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A modified ligand gated ion channel (LGIC) comprising at least one modified LGIC subunit, the modified LGIC subunit comprising:
(a) a modified alpha7 nicotinic acetylcholine receptor (a7-nAChR) ligand binding domain (LBD), wherein the modified a7-nAChR LBD comprises: (i) an amino acid sequence having at least 93 percent sequence identity to a sequence set forth in SEQ ID NO: 1, SEQ ID NO: 2, or SEQ ID NO:3, and (ii) an amino acid substitution at one or more of amino acid residues 154, 155, 156, 163, and 172 as numbered in SEQ ID NO:1, SEQ ID NO:2, and SEQ ID NO:3, and
(b) an ion pore domain (IPD).

2. The modified LGIC of claim 1, wherein the amino acid substitution at residue 154 is selected from the group consisting of V154I and V154L.

3. The modified LGIC of claim 1, wherein the amino acid substitution at residue 155 is R155Y.

4. The modified LGIC of claim 1, wherein the amino acid substitution at residue 156 is selected from the group consisting of W156Q and W156N.

5. The modified LGIC of claim 1, wherein the amino acid substitution at residue 163 is selected from the group consisting of H163T, H163A, H163D, H162F, H163G, H163K, H163N, H163R, H163S, and H163V.

6. The modified LGIC of claim 1, wherein the amino acid substitution at residue 172 is selected from the group consisting of S172A, S172V, and S172I.

7. The modified LGIC of claim 1, wherein the modified a7-nAChR LBD further comprises an amino acid substitution at residue 131 of the α7-nAChR LBD.

8. The modified LGIC of claim 7, wherein the amino acid substitution at residue 131 is selected from the group consisting of L131A, L131G, L131M, and L131N.

9. The modified LGIC of claim 1, wherein the modified a7-nAChR LBD further comprises an amino acid substitution at residue 139 of the α7-nAChR LBD.

10. The modified LGIC of claim 9, wherein the amino acid substitution at residue 139 is selected from the group consisting of Q139G and Q139L.

11. The modified LGIC of claim 1, wherein the modified a7-nAChR LBD further comprises an amino acid substitution at residue 217 of the α7-nAChR LBD.

12. The modified LGIC of claim 11, wherein the amino acid substitution at residue 217 is Y217F.

13. The modified LGIC of claim 1, wherein the modified a7-nAChR LBD further comprises a L131G amino acid substitution, a Q139L amino acid substitution, and a Y217F amino acid substitution.

14. The modified LGIC of claim 1, wherein the IPD is a murine 5HT3 IPD, and wherein the murine 5HT3 IPD further comprises an amino acid substitution of at least one of amino acid residues 425, 429, and 433.

15. The modified LGIC of claim 14, wherein the murine 5HT3 IPD comprises a R425Q substitution, a R429D substitution, and/or a R433A substitution.

16. The modified LGIC of claim 1, wherein the IPD is a human 5HT3 IPD, and wherein the human 5HT3 IPD further comprises an amino acid substitution of at least one of amino acid residues 420, 424, and 428.

17. The modified LGIC of claim 16, wherein the IPD is a human 5HT3 IPD comprises a R420Q substitution, a R424D substitution, and/or a R428A substitution.

18. A method of treating a channelopathy in a mammal, the method comprising:
administering to a cell in the mammal nucleic acid encoding the modified LGIC subunit of claim 1 under conditions in which the modified LGIC subunit can assemble into a modified LGIC comprising the modified LGIC subunit in a cell within the mammal; and
administering an exogenous ligand to the mammal, wherein the LGIC ligand acts as an agonist of the modified LGIC.

19. The method of claim 18, wherein the mammal is a human.

20. The method of claim 18, wherein the channelopathy is selected from the group consisting of Bartter syndrome, Brugada syndrome, catecholaminergic polymorphic ventricular tachycardia (CPVT), congenital hyperinsulinism, cystic fibrosis, Dravet syndrome, episodic ataxia, erythromelalgia, generalized epilepsy, familial hemiplegic migraine, fibromyalgia, hyperkalemic periodic paralysis, hypokalemic periodic paralysis, Lambert-Eaton myasthenic syndrome, long QT syndrome, short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, and multiple sclerosis.

21. A method of modulating ion transport across a cell membrane of a mammalian cell, the method comprising:
administering to the cell nucleic acid encoding the modified LGIC subunit of claim 1 under conditions in which the modified LGIC subunit can assemble into a modified LGIC comprising the modified LGIC subunit within the cell; and
administering an exogenous ligand to the cell, wherein the LGIC ligand acts as an agonist of the modified LGIC.

22. The method of claim 21, wherein the modulating comprises activating ion transport.

23. The method of claim 21, wherein the modulating comprises inhibiting ion transport.

24. The method of claim 21, wherein the cell is selected from the group consisting of a neuron, a glial cell, a myocyte, a stem cell, an endocrine cell, and an immune cell.

25. The method of claim 21, wherein the nucleic acid encoding the modified LGIC subunit is administered to the cell in vivo.

26. The method of claim 21, wherein the nucleic acid encoding the modified LGIC subunit is administered to the cell ex vivo.

27. The method of claim 21, wherein the mammalian cell is a human cell.

28-32. (canceled)

33. A mammalian cell comprising the modified LGIC of claim 1.

34. The mammalian cell of claim 33, wherein the mammalian cell is a human cell.

35. A nucleic acid encoding expressing the modified LGIC subunit of claim 1.

36-42. (canceled)

43. As isolated cell in culture comprising a nucleic acid encoding a modified ligand gated ion channel (LGIC) comprising at least one modified LGIC subunit, the modified LGIC subunit comprising:
(a) a modified alpha7 nicotinic acetylcholine receptor (a7-nAChR) ligand binding domain (LBD), wherein the modified a7-nAChR LBD comprises: (i) an amino acid sequence having at least 93% sequence identity to a sequence set forth in SEQ ID NO:1, SEQ ID NO:2, or SEQ ID NO:3, and (ii) an amino acid substitution at one or more of
amino acid residues 154, 155, 156, 163, and 172 as numbered in SEQ ID NO:1, SEQ ID NO: 2, and SEQ ID NO:3, and
(b) an ion pore domain (IPD).


SEQUENCE LISTING (21 sequences)

SEQ ID NO: 1 - protein, 224 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTV

SEQ ID NO: 2 - protein, 229 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRR

SEQ ID NO: 3 - protein, 233 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYY

SEQ ID NO: 4 - protein, 502 aa, Rattus rattus
MGGGRGGIWLALAAALLHVSLQGEFQRRLYKELVKNYNPLERPVANDSQPLTVYFSLSLLQIMDVDEKNQVLTTNIWLQMSWTDHYLQWNMSEYPGVKNVRFPDGQIWKPDILLYNSADERFDATFHTNVLVNASGHCQYLPPGIFKSSCYIDVRWFPFDVQQCKLKFGSWSYGGWSLDLQMQEADISSYIPNGEWDLMGIPGKRNEKFYECCKEPYPDVTYTVTMRRRTLYYGLNLLIPCVLISALALLVFLLPADSGEKISLGITVLLSLTVFMLLVAEIMPATSDSVPLIAQYFASTMIIVGLSVVVTVIVLRYHHHDPDGGKMPKWTRIILLNWCAWFLRMKRPGEDKVRPACQHKPRRCSLASVELSAGAGPPTSNGNLLYIGFRGLEGMHCAPTPDSGVVCGRLACSPTHDEHLMHGAHPSDGDPDLAKILEEVRYIANRNRCQDESEVICSEWKFAACVVDPLCLMAFSVFTIICTIGILMSAPNFVEAVSKDFA

SEQ ID NO: 5 - protein, 247 aa, Mus musculus
IIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKRDEMREVARDWLRVGYVLDRLLFRIYLLAVLAYSITLVTLWSIWHYS

SEQ ID NO: 6 - protein, 242 aa, Homo sapiens
IIRRRPLFYVVSLLLPSIFLMVMDIVGFYLPPNSGERVSFKITLLLGYSVFLIIVSDTLPATAIGTPLIGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 7 - protein, 471 aa, Synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVIIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKRDEMREVARDWLRVGYVLDRLLFRIYLLAVLAYSITLVTLWSIWHYS

SEQ ID NO: 8 - protein, 466 aa, Synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVIIRRRPLFYVVSLLLPSIFLMVMDIVGFYLPPNSGERVSFKITLLLGYSVFLIIVSDTLPATAIGTPLIGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 9 - protein, 7 aa, Synthetic construct
FCYENEV

SEQ ID NO: 10 - protein, 21 aa, Synthetic construct
MRRAPSLVLFFLVALCGRGNC

SEQ ID NO: 11 - protein, 65 aa, Synthetic construct
QSQPILNTKEMAPQSKPPEELEMSSMPSPVAPLPARTEGVIDMRSMSSIDSFISCATDFPEATRF

SEQ ID NO: 12 - DNA, 1509 bp, Rattus rattus
  source 1..1509: mol_type = genomic DNA
atgggcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaataactgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcaaccgctgccaggacgagagtgaggtgatctgcagtgaatggaagtttgcagcctgcgtggtggacccgctttgcctcatggccttttcggtctttaccatcatctgtaccatcggcatcctcatgtcagctccaaactttgtggaggctgtgtccaaagactttgcttaa

SEQ ID NO: 13 - DNA, 672 bp, Homo sapiens
  source 1..672: mol_type = genomic DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaaccctaccctgacgtaacattcacagtt

SEQ ID NO: 14 - DNA, 687 bp, Synthetic construct
  source 1..687: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagccctacccagatgtcaccttcacagtgaccatgcggagacgc

SEQ ID NO: 15 - DNA, 699 bp, Homo sapiens
  source 1..699: mol_type = genomic DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagccctacccagatgtcaccttcacagtgaccatgcggagacgcacactgtattac

SEQ ID NO: 16 - DNA, 744 bp, Mus musculus
  source 1..744: mol_type = genomic DNA
atcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcgggacgagatgagggaagtcgctagagactggctgcgagtgggctacgtccttgacaggctgctgtttcggatctacttgctggcggtgctggcttattccattactctggtgacactctggtccatatggcactacagttag

SEQ ID NO: 17 - DNA, 726 bp, Homo sapiens
  source 1..726: mol_type = genomic DNA
atcatccgtagaaggcctctgttttacgtggtgagcctgctgctgccatccatcttcctgatggtcatggacatcgtgggcttttacctgccacccaattctggcgagcgcgtgagcttcaagatcacactgctgctgggctatagcgtgtttctgatcatcgtgtccgataccctgcctgcaacagcaatcggaaccccactgatcggcgtgtatttcgtggtgtgcatggccctgctggtcatcagcctggccgagacaatctttatcgtgcggctggtgcacaagcaggacctgcagcagcctgtgccagcatggctgaggcacctggtgctggagaggatcgcatggctgctgtgcctgagagagcagtccacatctcagaggcctccagccacctctcaggccaccaagacagacgattgctctgccatgggcaatcactgtagccacatgggcggcccccaggactttgagaagtcccctcgcgatcggtgctctccacctccaccacctagggaggccagcctggccgtgtgcggcctgctgcaggagctgtcctctatccggcagttcctggagaagcgcgacgagatccgggaggtggccagagattggctgagggtgggcagcgtgctggataagctgctgtttcacatctacctgctggcagtcctggcctattctattaccctggtcatgctgtggtccatctggcagtacgcc

SEQ ID NO: 18 - DNA, 1416 bp, Synthetic construct
  source 1..1416: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaaccctaccctgacgtaacattcacagttatcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcgggacgagatgagggaagtcgctagagactggctgcgagtgggctacgtccttgacaggctgctgtttcggatctacttgctggcggtgctggcttattccattactctggtgacactctggtccatatggcactacagttag

SEQ ID NO: 19 - DNA, 21 bp, Synthetic construct
  source 1..21: mol_type = other DNA
ttttgctatgaaaacgaagtc

SEQ ID NO: 20 - DNA, 63 bp, Synthetic construct
  source 1..63: mol_type = other DNA
atgagaagggccccatccctggtattgttttttttggtagctttgtgcgggagggggaactgc

SEQ ID NO: 21 - DNA, 195 bp, Synthetic construct
  source 1..195: mol_type = other DNA
caaagccaacctatccttaacactaaagagagcgccgctcaatccaaacccaaagaagagttggaaatggagtctataccttcacctgttgcacctctccctactaggaccgaaggcgtgattgacatgcgctctatgtctagtatagatagctttatatcctgcgccacagactttcccgaagccactaggttc